(19) United States
(12) Patent Application Publication
(10) Pub. No.: US 2024/0191252 A1
(43) Pub. Date: Jun. 13, 2024

(54) GENETICALLY MODIFIED ONCOLYTIC HERPES SIMPLEX VIRUS DELIVERING CHEMOKINE AND TUMOR ASSOCIATED/SPECIFIC ANTIGEN

(71) Applicant: IMMVIRA BIOPHARMACEUTICALS CO., LIMITED, Suzhou, Jiangsu (CN)

(72) Inventors: Xiaoqing CHEN, Shenzhen (CN); Yuanyuan LIU, Shenzhen (CN); Ziwen LIU, Dongguan (CN); Tianyi DENG, Shenzhen (CN); Grace Guoying ZHOU, Shenzhen (CN)

(21) Appl. No.: 18/286,103

(22) PCT Filed: Apr. 7, 2022
(86) PCT No.: PCT/CN2022/085474

§ 371 Date: Oct. 6, 2023

(30) Foreign Application Priority Data
Apr. 8, 2021 (WO) PCT/CN2021/085930
Jul. 14, 2021 (WO) PCT/CN2021/106319

(51) Int. Cl. C12N 15/86; A61K 35/763; A61K 39/00; A61P 35/00; C07K 14/47; C07K 14/52; C07K 14/54; C07K 14/82; C07K 16/28

(52) U.S. Cl. CPC C12N 15/86; A61K 35/763; A61K 39/001106; A61K 39/001112; A61K 39/4611; A61K 39/4613; A61K 39/4631; A61K 39/464412; A61P 35/00; C07K 14/4748; C07K 14/521; C07K 14/5434; C07K 14/82; C07K 16/2818; A61K 2039/5256; A61K 2239/46; A61K 2239/57; A61K 2239/58; C12N 2710/16632; C12N 2710/16643

(57) ABSTRACT

Disclosed is a genetically modified oncolytic herpes simplex virus (oHISV) encoding a truncated nonsignaling variant of at least one tumor associated/specific antigen, and at least one chemokine. The expression of the truncated nonsignaling variant and the chemokine is under the control of an immediate-early gene promoter of HSV, and the truncated nonsignaling variant is expressed and presented on a tumor cell surface as a biomarker upon replication of the oHSV in the tumor cell, and the chemokine is expressed and released to induce chemotaxis of an immune cell towards the tumor cell. The genetically modified oHSV can be used in combination with CAR-T, ADC, and/or BiTE therapies.

### TECHNICAL FILED

The present disclosure is related to an oncolytic herpes simplex virus genetically engineered to carry genes encoding at least one chemokine and/or at least one tumor associated/specific antigen, and its use in combination with a tumor-targeting therapeutic agent including CAR-T, BiTE, and ADC for treatment of various tumors.

### BACKGROUND

Adoptive transfer of CAR-T cells in patients with hematologic malignancies has shown stunning results. However, this approach has shown little effect in patients with solid tumors. It appears unlikely that CAR-T cell therapy alone will be sufficient to induce complete responses in the majority of cancers. Combining CAR-T cells with other cancer treatments that have different mechanisms of action and the potential to synergize with T cells may reduce tumor escape and increase the success rates of CAR-T cell therapy.

Oncolytic virotherapy is a therapeutic approach to treat cancer that uses native or genetically modified viruses that selectively replicate within cancer cells. The field of oncolytic virotherapy has gained renewed attention after the FDA approval of Talimogene laherparepvec (T-VEC), an oncolytic herpes simplex virus type 1 (HSV-1) modified to express GM-CSF. Moreover, oncolytic viruses (OV) can be further modified to selectively deliver therapeutic transgenes to the tumor microenvironment to enhance their antitumor potency or boost an antitumor immune response. Preclinical studies combining CAR-T cells with oncolytic viruses armed with cytokines, chemokines, BiTEs, or immune checkpoint inhibitors resulted in enhanced therapeutic outcomes. For example, oncolytic adenoviruses modified to express IL-15 and RANTES or IL-2 and TNF-α have been shown to increase the accumulation and survival of CAR-T cells in the tumor microenvironment. A vaccinia virus expressing CXCL11, a CXCR3 ligand, was used to attract effector cells following transfer to enhance the intra-tumoral trafficking of CAR-T cells. Another report demonstrated that expression by an oncolytic adenovirus of a BiTE targeting a second tumor antigen could address heterogeneity of antigen expression. As expected, all these combinations of CAR-T cells and armed-OV resulted in enhanced tumor control and prolonged survival when compared to each agent as monotherapy.

A recent study engineered an oncolytic virus to express a nonsignaling, truncated CD19 (CD19t) protein for tumor-selective delivery, enabling targeting by CD19-CAR T cells. Infecting tumor cells with an oncolytic vaccinia virus coding for CD19t (OV19t) produced de novo CD19 at the cell surface before virus-mediated tumor lysis. Cocultured CD19-CAR T cells secreted cytokines and exhibited potent cytolytic activity against infected tumors. Using several mouse tumor models, delivery of OV19t promoted tumor control after CD19-CAR T cell administration. OV19t induced local immunity characterized by tumor infiltration of endogenous and adoptively transferred T cells. CAR T cell-mediated tumor killing also induced release of virus from dying tumor cells, which propagated tumor expression of CD19t. While the study cured more than 50% of mice treated with this combination therapy, some mice either responded transiently or did not respond (Park et al., Sci. Transl. Med. 12, eaaz1863 (2020)).

US20190233536A1 disclosed a modified adenovirus, in particular Enadenotucirev (EnAd), armed with at least two bispecific T cell engager (BiTE) each comprising at least two binding domains, wherein at least one of the domains is specific for a surface antigen on an immune cell of interest, such as a T-cell of interest. Arming an adenovirus with a BiTE molecule allows the bi-specific antibody fragment molecule to ‘piggyback’ on the ability of the adenovirus to selectively infect cancer cells, thereby enabling the targeted delivery of the BiTE to tumor cells. Once infected by the adenoviruses, the BiTE molecules are synthesized by tumor cells, secreted and can act locally, spreading beyond the immediate footprint of the virus. This therefore allows the BiTE to spread beyond the immediate site of infection but at the same time limits the spread of the virus too far beyond the infected tumor cell nest. This minimizes the risk of undesired off-target effects.

### SUMMARY

A first aspect of the disclosure is related to a genetically modified oncolytic herpes simplex virus (oHSV), wherein the genome of the oHSV is incorporated with a polynucleotide encoding (a) a truncated nonsignaling variant of at least one tumor associated/specific antigen, and (b) at least one chemokine, wherein the expression of the truncated nonsignaling variant and the at least one chemokine is under the control of an immediate-early gene promoter of HSV, and wherein the truncated nonsignaling variant is expressed and presented on a tumor cell surface as a biomarker upon replication of the oHSV in the tumor cell, and the at least one chemokine is expressed and released to induce chemotaxis of an immune cell towards the tumor cell.

Another aspect of the disclosure is related to a genetically modified oncolytic herpes simplex virus (oHSV), wherein the genome of the oHSV is incorporated with a polynucleotide encoding a truncated nonsignaling variant of at least one tumor associated/specific antigen, wherein the expression of the truncated nonsignaling variant is under the control of an immediate-early gene promoter of HSV, and wherein the truncated nonsignaling variant is expressed and presented on a tumor cell surface as a biomarker upon replication of the oHSV in the tumor cell.

Another aspect of the disclosure is related to a pharmaceutical kit for treatment of a cancer, comprising, separately, a genetically modified oncolytic herpes simplex virus (oHSV) as described herein and a tumor-targeting therapeutic agent, wherein the tumor-targeting therapeutic agent has a targeting moiety specific to the truncated nonsignaling variant of the at least one tumor associated/specific antigen encoded by the polynucleotide, and an effector moiety for killing or inhibiting the proliferation of a cell of the cancer.

A further aspect of the disclosure is related to a method for treatment of a cancer in a subject, comprising administering to the subject a pharmaceutically effective amount of a genetically modified oncolytic herpes simplex virus (oHSV) and a tumor-targeting therapeutic agent, concurrently or sequentially, wherein the genome of the oHSV is incorporated with a polynucleotide encoding (a) a truncated nonsignaling variant of at least one tumor associated/specific antigen, and preferably, (b) at least one chemokine, wherein the expression of the truncated nonsignaling variant and preferably, the at least one chemokine, is under the control of an immediate-early gene promoter of HSV, and wherein the tumor-targeting therapeutic agent has a targeting moiety specific to the truncated nonsignaling variant of the at least one tumor associated/specific antigen encoded by the polynucleotide, and an effector moiety for killing or inhibiting the proliferation of a cell of the cancer.

Further aspects of the disclosure will be readily seen from the detailed description as described in the following in reference to the drawings.

### BRIEF DESCRIPTION OF DRAWINGS

FIG. 1 shows schematic diagram of the oHSV backbones of T7201, T7202, T7203, T7204, T7011, T7012, and T7013 (collectively referred to as “T7 series” hereinafter). (A) A schematic diagram of T3011, a genetically modified oHSV encodes hPD-1-Antibody (hPD-1-Ab), an immune checkpoint inhibitor, and hIL-12, a cytokine, with the internal inverted repeat (b′a′ and a′c′) replaced by the polynucleotides encoding hIL-12 and the expression cassette of hPD-1-Ab is introduced between genes UL3 and UL4 of the UL fragment. A more detailed description of T3011 can be found in WO 2017/181420 (IMMV503), and the entire disclosure of which is incorporated herein by reference. (B) A schematic diagram of exemplary T7 series viruses described herein. These genetically modified oHSVs encode hPD-1-Ab, hIL-12, a tumor associated antigen, and a chemokine, with the internal inverted repeat (b′a′ and a′c′) replaced by the polynucleotides encoding hIL-12, the expression cassette of hPD-1-Ab introduced between genes UL3 and UL4 of the UL fragment, and the TAA+Chemokine expression cassette introduced between genes UL37 and UL38 of the UL fragment. (C) A schematic diagram of exemplary genetically modified oHSVs T7201, T7202, T7203 and T7204, in which the TAA+Chemokine expression cassette in inset B is embodied as one TAA (truncated non-signaling variant of Tumor Associated Antigen) and one chemokine. (D) A schematic diagram of exemplary genetically modified oHSVs T7011, T7012, and T7013, in which the TAA+Chemokine expression cassette in inset B is specified as two different TAAs plus one chemokine, designated as TAA1+TAA2+chemokine. An HSV-IE (Immediate-Early) promoter and a PolyA tail are located upstream and downstream the expression cassette, respectively.

FIG. 2 shows the flowchart of construction of the T7 series oHSV (i.e., T7011 to T7013, and T7201 to T7204, as described above). The construction involved several steps of cloning with the aid of bacterial artificial chromosome (BAC) system.

FIG. 3 shows the CCL5 release after T7011 infection in 293T, HEp-2, and Tca8113 cells. The expression and release of CCL5 was fast and strong. Secreted CCL5 was detected as soon as 4 hours post-infection and peaked as high as 5,000 pg/ml. The secretion was stable and retained for up to at least 4 days after T7011 viral infection. It thus showed that the CCL5 secretion.

FIG. 4 shows the expression of truncated CD19, BCMA, Trop-2, HER2 on cell surface. The different truncated antigens encoded by T7011, T7012, and T7013, respectively, were concurrently expressed on tumor cell surfaces.

FIG. 5 shows anti-tumor effect of T7 series (T7011, T7012, and T7013) oHSV virus. The IC₅₀ values of T7 series oHSV virus are comparable to T3011, indicating that T7 series viruses have similar broad anti-tumor activity compared to T3011.

FIG. 6 shows T7 series (T7011, T7012, and T7013) oHSV virus have no infectious activity in either CAR-T or normal T Cells.

FIG. 7 shows T7 series (T7011, T7012, and T7013) oHSV virus have no cell killing activity in either CAR-TCD19 or normal T Cells.

FIG. 8 shows the anti-tumor effect is significantly enhanced in T7011 and CAR-TCD19 combo treatment.

FIG. 9 shows T7011 virus infection can specifically synergize CAR-TCD19 anti-tumor activity.

FIG. 10 shows the anti-tumor effect is significantly enhanced in T7012 and CAR-TCD19 combo treatment.

FIG. 11 shows T7012 virus infection can specifically synergize CAR-TCD19 anti-tumor activity.

FIG. 12 shows the anti-tumor effect is significantly enhanced in T7013 and CAR-TCD19 combo treatment.

FIG. 13 shows T7013 virus infection can specifically synergize CAR-TCD19 anti-tumor activity.

FIG. 14 shows T7 series (T7011, T7012, and T7013) oHSV virus have no cell killing ability in either CAR-NKCD19 or NK Cells.

FIG. 15 shows viral replication of HSV-1 (F) and T7011 in CAR-NKCD19 and NK cells.

FIG. 16 shows T7011 has no negative effect on CAR-NKCD19 cell proliferation. * p<0.05, *** p<0.001.

FIG. 17 shows T7011 has no negative effect on NK cell proliferation. * p<0.05, ** p<0.01, *** p<0.001.

FIG. 18 shows the anti-tumor effect is significantly enhanced in T7011 and CAR-NKCD19 combo treatment.

FIG. 19 shows T7011 virus infection can specifically synergize CAR-NKCD19 anti-tumor activity.

### DETAILED DESCRIPTION OF THE INVENTION

### Definition

It is to be noted that the term “a” or “an” entity refers to one or more of that entity; for example, “a truncated nonsignaling variant” is understood to represent one or more truncated nonsignaling variants. As such, the terms “a” (or “an”), “one or more,” and “at least one” can be used interchangeably herein.

The terms “antibody fragment” or “antigen binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgGI, IgG2, IgG3, IgG4, IgA1 and IgA2) or subclass of immunoglobulin molecule. For example, an anti-PD-1 antibody may refer to an antigen binding fragment thereof, such as, Fab fragment or scFv thereof.

By “specifically binds”, “specific to”, or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, “cancer” or “tumor” as used interchangeably herein is meant to a group of diseases which can be treated according to the disclosure and involve abnormal cell growth with the potential to invade or spread to other parts of the body. Not all tumors are cancerous; benign tumors do not spread to other parts of the body. Possible signs and symptoms include: a new lump, abnormal bleeding, a prolonged cough, unexplained weight loss, and a change in bowel movements among others. There are over 100 different known cancers that affect humans. As used herein, “cancer” includes, without limitation, a solid cancer (e.g., a tumor) and a hematologic malignancy. A “hematologic malignancy”, also known as a blood cancer, is a cancer that originates in blood-forming tissue, such as the bone marrow or other cells of the immune system. Hematologic malignancies include, without limitation, leukemias (such as acute myeloid leukemia (ANIL), acute promyelocytic leukemia, acute lymphoblastic leukemia (ALL), acute mixed lineage leukemia, chronic myeloid leukemia, chronic lymphocytic leukemia (CLL), hairy, cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma, non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies. “Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an oHSV-1 or composition of the present disclosure used, e.g., for detection, for a diagnostic procedure and/or for treatment.

It will also be understood by one of ordinary skill in the art that modified genomes as disclosed herein may be modified such that they vary in nucleotide sequence from the modified polynucleotides from which they were derived. For example, a polynucleotide or a nucleotide sequence derived from a designated DNA sequence may be similar, e.g., have a certain percent identity to the starting sequence, e.g., it may be 60%, 70%, 75%, 80%, 85%, 90%, 95%, 98%, or 99% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

“Therapeutically effective amount” or “effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

A CAR-T cell is a T cell which expresses a chimeric antigen receptor. The T cell expressing a CAR molecule may be a helper T cell, a cytotoxic T cell, a viral-specific cytotoxic T cell, a memory T cell, or a gamma delta (γδ) T cell. A chimeric antigen receptor (CAR), is a recombinant fusion protein comprising: 1) an extracellular ligand-binding domain, i.e., an antigen-recognition domain, 2) a transmembrane domain, and 3) a signaling transducing domain. The extracellular ligand-binding domain is an oligo- or polypeptide that is capable of binding a ligand. Preferably, the extracellular ligand-binding domain will be capable of interacting with a cell surface molecule which may be an antigen, a receptor, a peptide ligand, a protein ligand of the target, or a polypeptide of the target. In the present disclosure, the extracellular ligand-binding domain will be capable of interacting with the truncated nonsignaling variant of a tumor associated antigen or a tumor specific antigen.

Typically, the extracellular ligand-binding domain is linked to the signaling transducing domain of the chimeric antigen receptor (CAR) by a transmembrane domain I. The transmembrane domain traverses the cell membrane, anchors the CAR to the T cell surface, and connects the extracellular ligand-binding domain to the signaling transducing domain, impacting the expression of the CAR on the T cell surface. The transmembrane domain can further comprise a hinge region between extracellular ligand-binding domain and said transmembrane domain. The term “hinge region” generally means any oligo- or polypeptide that functions to link the transmembrane domain to the extracellular ligand-binding domain. In particular, hinge region is used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or parts of naturally-occurring molecules such as CD28, 4-1BB (CD137), OX-40 CD134), CD3ζ, the T cell receptor α or β chain, CD45, CD4, CD5, CD8, CD8α, CD9, CD16, CD22, CD33, CD37, CD64, CD80, CD86, ICOS, CD154 or from all or parts of an antibody constant region. Alternatively, the hinge region may be a synthetic sequence that corresponds to a naturally-occurring hinge sequence or the hinge region may be an entirely synthetic hinge sequence.

A chimeric antigen receptor (CAR) further comprises a signal transducing domain or intracellular signaling domain of a CAR which is responsible for intracellular signaling following the binding of the extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper T cell activity, including the secretion of cytokines. Thus, the term “signal transducing domain” refers to the portion of a protein which transduces the effector signal function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Non-limiting examples of ITAM that can be used in the present disclosure can include those derived from TCRζ, FcRγ, FcRβ, FcRε, CD3γ, CD3δ, CD3ε, CDS, CD22, CD79a, CD79b and CD66d. In one embodiment, the signaling transducing domain of the CAR can comprise the CD3ζ signaling domain with an amino acid sequence of at least 80%, 90%, 95%, 97%, or 99% sequence identity thereto. The present disclosure envisages the combinational use of the genetically engineered oHSVs described herein with any CAR-T without limitation.

Typical antibody-drug conjugates (ADC) contain monoclonal antibodies capable to bind to surface specific antigens of cancer cells. These antibodies include some proteins on the surface of immune system B cells and T cells, such as CD20, CD22, and human epidermal growth factor receptor 2 (Her2) and prostate specific membrane antigen (PSMA). These antibodies are connected with the highly toxic drugs through a cleavable linker unit. Drugs are designed to induce irreversible DNA damage or interfere with cell division, so as to lead to apoptosis of cancer cells. ADCs contain monoclonal antibodies capable to bind to surface specific antigens of cancer cells. These antibodies include some proteins on the surface of immune system B cells and T cells, such as CD20, CD22, and human epidermal growth factor receptor 2 (Her2) and prostate specific membrane antigen (PSMA). These antibodies are connected with the highly toxic drugs through a cleavable linker unit. Drugs are designed to induce irreversible DNA damage or interfere with cell division, so as to lead to apoptosis of cancer cells.

The mechanism of antibody drug conjugates (ADC) is to recognize and bind to specific antigen through the antibodies, trigger a series of reactions, and then enter the cytoplasm through the endocytosis, where the highly toxic drug is dissociated from the lysosomal enzymes to kill cancer cells. Compared with the traditional chemotherapy which causes damage to both cancer cells and normal tissues indiscriminately, targeting drug delivery can make the drug act on cancer cells directly and reduce the damage to normal cells. Typical antibody drug conjugate is comprised by three parts of a drug, a linker unit and an antibody. The choice of specific antibody and drug depends on specific diseases and has important effects on the safety and efficacy of the conjugate. The stability of the linker unit and the coupling method to the antibody play a decisive role in the development of ADC drug. Factors that determine the efficacy of antibody drug conjugates include the stability and fracture sensitivity of the linker unit, cell surface excitation internalization, transport, and release of the cytotoxin. The present disclosure envisages the combinational use of the T7 series oHSVs described herein with any ADC without limitation.

Bispecific T-cell engagers (BiTEs) are relatively simple bi-specific molecules that are specific for the CD3 E subunit of the TCR complex of a T-cell and also a target an antigen of interest, such as a cancer antigen. Since BiTEs are specific for the TCR complex, this enables BiTEs to activate resident T-cells to kill cells expressing a particular target antigen on their cell surface, for example cancer cells. An important property of BiTEs is their ability to make CD4+ and non-activated CD8+ T-cells target cancer cells. In other words, T-cells activated by BiTES can be made to kill cells independent of MHC expression on the cell surface. This is important because some tumor cells downregulate MHC which makes them resistant to agents such as CAR-T cells and immTACs. Unfortunately, BiTEs have poor circulation kinetics relative to full length antibodies. This means that when administered to the patient, a large proportion of the BiTEs do not reach their target cells. In addition, the use of high affinity anti-CD3 ScFv as part of the BiTE can lead to strong binding to T-cells in the blood, which also interferes with delivery to the tumor. As a result, the BiTEs are unable to reach their full potential as an anti-cancer therapy because they cannot be effectively delivered to the tumor cells. The present disclosure envisages the combinational use of the oHSV described herein with any BiTEs without limitation.

As used herein, the term “tumor associated/specific antigen” is meant tumor associated antigen, tumor specific antigen, or both. For example, the term “at least one tumor associated/specific antigen” means at least one tumor associated antigen or at least one tumor specific antigen, and may include a pair of a tumor associated antigen and a tumor specific antigen. For example, the term “a truncated nonsignaling variant of at least one tumor associated/specific antigen” is intended to include a truncated non-signaling variant of one tumor associated antigen, truncated non-signaling variants of two or more tumor associated antigens, a truncated non-signaling variant of one tumor specific antigen, truncated non-signaling variants of two or more tumor specific antigens, truncated non-signaling variants of one tumor associated antigen and two or more tumor specific antigens, and truncated non-signaling variants of two or more tumor associated antigens and one tumor specific antigen. For example, the term “two tumor associated/specific antigens” may include two tumor associated antigens, two tumor specific antigens, and a combination of one tumor associated antigen and one tumor specific antigen.

As used herein, “biomarker”, “a truncated nonsignaling variant”, “a truncated variant” or “a nonsignaling variant” of a specific tumor associated antigen or tumor specific antigen, refers to a variant of the tumor associated antigen or tumor specific antigen that is mutated, deleted or otherwise modified to disable the signal transduction of its wild-type counterpart in a signaling pathway. The variant exposes at least some of the epitopes of the antigen such that the variant is able to be bound by an antibody or an antigen binding fragment thereof (such as a scFv) specifically directed against the wild-type antigen. A commonly known nonsignaling variant of a tumor associated antigen or tumor specific antigen, in the case that the antigen is a transmembrane protein, is an extracellular domain of the antigen, an extracellular-transmembrane domain of the antigen, or an equivalent thereof that has at least 90% amino acid sequence identity to the extracellular or extracellular-transmembrane domain. The equivalent is not able to transduce signal but exposes at least some of epitopes on the extracellular domain of the antigen.

For example, a nonsignaling variant of CD19 (also referred to as a nonsignaling CD19 herein) is a 323-aa extracellular-transmembrane domain (SEQ ID NO: 14) of wild-type CD19. A nonsignaling BCMA is a 77-aa extracellular-transmembrane domain (SEQ ID NO: 15) of wild-type BCMA. A nonsignaling HER2 is a 675-aa extracellular-transmembrane domain (SEQ ID NO: 16) of wild-type HER2. A nonsignaling Trop-2 is a 297-aa extracellular-transmembrane domain (SEQ ID NO: 17) of wild-type Trop-2.

The extracellular and transmembrane domains can be obtained by routine practice in the art without difficulties. The amino acid sequences of the extracellular/transmembrane domains of various tumor associated antigens or tumor specific antigens are available from public resources including NCBI (https://www.ncbi.nlm.nih.gov/protein). It is noted that the nonsignaling variants, once expressed on the tumor cell surface, are recognized and bound by an antibody or an antigen binding fragment of the antibody specific to the tumor associated antigen or tumor specific antigen. The antigen binding fragment may be a part of CAR-immune cell (e.g., a CAR-T cell or a CAR-NK cell) or BiTE. The antibody may be conjugated with a chemotherapeutic drug to form an ADC. However, the nonsignaling variant would not trigger a signaling pathway as its wild-type counterpart. A person skilled in the art would be easy to test and verify whether a variant of a tumor associated antigen or tumor specific antigen is nonsignaling. For example, it could be determined by detection of the level of a downstream protein in the normal signaling pathway known for the wild type antigen.

Genetically Modified Oncolytic Herpes Simplex Virus (oHSV)

The present disclosure provides a genetically modified oncolytic herpes simplex virus (oHSV). The genetically modified oHSV is modified such that it expresses a truncated nonsignaling variant of at least one tumor associated antigen or tumor specific antigen upon replication in a susceptible cell, for example, a solid tumor cell. The present inventors showed the successful expressions of different truncated tumor associated antigens or tumor specific antigens on the cell surfaces after the infection and replication of genetically modified oHSVs in the tumor cell. The nonsignaling tumor associated antigen or tumor specific antigen expressed and then presented onto the surface of the tumor cell labels that tumor cell as a target for various antigen directed therapies, such as CAR-T therapies. In some embodiments, the genetically modified oHSV of the present disclosure is further modified such that it expresses at least one chemokine upon replication in a susceptible cell, for example, a solid tumor cell. The present inventors showed the detection of secreted chemokine as soon as 4 hours post-infection and it was retained for up to at least 4 days after oHSV viral infection. The expression and release of the chemokine induces chemotaxis of an immune cell, for example, a T cell or CAR T cell, towards the susceptible cell, which facilitates trafficking and infiltration of the immune cell into the tumor mass.

In some embodiments, a genetically modified oncolytic herpes simplex virus (oHSV) is provided, wherein the genome of the oHSV is incorporated with a polynucleotide encoding a truncated nonsignaling variant of at least one tumor associated/specific antigen, wherein the expression of the truncated nonsignaling variant is under the control of an immediate-early gene promoter of HSV.

In some embodiments, a genetically modified oncolytic herpes simplex virus (oHSV) is provided, wherein the genome of the oHSV is incorporated with a polynucleotide encoding (a) a truncated nonsignaling variant of at least one tumor associated/specific antigen, and (b) at least one chemokine, wherein the expression of the truncated nonsignaling variant and the at least one chemokine is under the control of an immediate-early gene promoter of HSV.

In some embodiments, a genetically modified oncolytic herpes simplex virus (oHSV) is provided, wherein the genome of the oHSV is incorporated with a polynucleotide encoding (a) a truncated nonsignaling variant of one tumor associated antigen or one tumor specific antigen, and (b) one chemokine, wherein the expression of the truncated nonsignaling variant and the chemokine is under the control of an immediate-early gene promoter of HSV.

In some embodiments, a genetically modified oncolytic herpes simplex virus (oHSV) is provided, wherein the genome of the oHSV is incorporated with a polynucleotide encoding (a) truncated nonsignaling variants of two tumor associated/specific antigens, and (b) one chemokine, wherein the expression of the truncated nonsignaling variants and the chemokine is under the control of an immediate-early gene promoter of HSV. In some embodiments, the two tumor associated/specific antigens comprise two tumor associated antigens, which are same or different. In some embodiments, the two tumor associated/specific antigens comprise two tumor specific antigens, which are same or different. In some embodiments, the two tumor associated/specific antigens include one tumor associated antigen and one tumor specific antigen.

In some embodiments, a genetically modified oncolytic herpes simplex virus (oHSV) is provided, wherein the genome of the oHSV is incorporated with a polynucleotide encoding (a) a truncated nonsignaling variant of at least one tumor associated/specific antigen, and (b) two chemokines, wherein the expression of the truncated nonsignaling variant and the chemokines is under the control of an immediate-early gene promoter of HSV. In some embodiments, the two chemokines are same. In some embodiments, the two chemokines are different.

In some embodiments, a genetically modified oncolytic herpes simplex virus (oHSV) is provided, wherein the genome of the oHSV is incorporated with a polynucleotide encoding (a) truncated nonsignaling variants of two tumor associated/specific antigens, and (b) two chemokines, wherein the expression of the truncated nonsignaling variants and the chemokines is under the control of an immediate-early gene promoter of HSV. In some embodiments, the two chemokines are same. In some embodiments, the two chemokines are different. In some embodiments, the two tumor associated/specific antigens comprise two tumor associated antigens, which are same or different. In some embodiments, the two tumor associated/specific antigens comprise two tumor specific antigens, which are same or different. In some embodiments, the two tumor associated/specific antigens include one tumor associated antigen and one tumor specific antigen.

Thus, in some embodiments of the disclosure, a genetically modified oHSV is incorporated into its genome with a first polynucleotide that encodes a truncated nonsignaling variant of at least one tumor associated/specific antigen, and a second polynucleotide that encodes at least one chemokine, wherein the expression of the truncated nonsignaling variant and the at least one chemokine is under the control of an immediate-early gene promoter of HSV.

In some embodiments, the genetically modified oHSV is incorporated into its genome with a first polynucleotide that encodes a truncated nonsignaling variant of a first tumor associated/specific antigen, a second polynucleotide that encodes a truncated nonsignaling variant of a second tumor associated/specific antigen, and a third polynucleotide that encodes a chemokine, wherein the expression of the truncated nonsignaling variants and the chemokine is under the control of an immediate-early gene promoter of HSV.

In some embodiments, the tumor associated/specific antigen, the first or second tumor associated/specific antigen, is independently selected from a group consisting of HER2, PSMA, BCMA, CD20, CD33, CD19, CD22, CD123, CD30, GPC-3, CEA, Claudin18.2, EpCAM, GD2, MSLN, EGFR, MUC1, EGFRVIII, CD38, Trop-2, c-MET, Nectin-4, CD79b, CCK4, GPA33, HLA-A2, CLEC12A, p-cadherin, TDO2, MART-1, Pmel 17, MAGE-1, AFP, CA125, TRP-1, TRP-2, NY-ESO, PSA, CDK4, BCA225, CA 125, MG7-Ag, NY-CO-1, RCAS 1, SDCCAG16, TAAL6 and TAG72. In some embodiments, the tumor associated/specific antigen is selected from a group consisting of HER2, Trop-2, BCMA, and CD19.

In some embodiments, the chemokine is selected from a group consisting of CXCL1 to CXCL17, CCL1 to CCL 28, XCL1, XCL2, and CX3CL1. In preferred embodiments, the chemokine is selected from a group consisting of CXCL9, CXCL10, CXCL11, CXCL12, CCL3, CCL4, CCL5, CCL19, CCL21. In preferred embodiments, the chemokine is CCL5.

In some embodiments, the immediate-early gene promoter of HSV is an immediate-early gene promoter of HSV-1 or HSV-2. In some embodiments, the immediate-early gene promoter of HSV is selected from a group consisting of IE 1 (ICP0 promoter), IE 2 (ICP27 promoter), IE 3 (ICP4 promoter), and IE 4/5 (ICP22 and ICP47 promoter) of HSV-1. In preferred embodiments, the immediate-early gene promoter of HSV is immediate-early gene promoter IE4/5 of HSV-1.

In some embodiments, the truncated nonsignaling variant is an extracellular-transmembrane domain of the tumor associated/specific antigen. For example, the truncated nonsignaling variant of CD19 is an extracellular-transmembrane domain of CD19. For example, the truncated nonsignaling variant of BCMA is an extracellular-transmembrane domain of BCMA. For example, the truncated nonsignaling variant of HER2 is an extracellular-transmembrane domain of HER2. For example, the truncated nonsignaling variant of Trop-2 is an extracellular-transmembrane domain of Trop-2. In some embodiments, the truncated nonsignaling variant is an extracellular domain of the tumor associated/specific antigen. In some embodiments, the truncated nonsignaling variant is an extracellular domain linked to a part of a transmembrane domain of the tumor associated/specific antigen. In some embodiments, the truncated nonsignaling variant is a variant of a wild-type tumor associated/specific antigen that is lack of a part of or a whole signal transduction domain.

In preferred embodiments, the genetically modified oHSV is originated from HSV type 1 (HSV-1) or HSV type 2 (HSV-2). In preferred embodiments, the genetically modified oHSV is originated from a F strain of HSV-1.

In preferred embodiments, the polynucleotide as described herein encodes (i) a truncated nonsignaling variant of CD19, and (ii) CCL5. In preferred embodiments, the polynucleotide encodes (i) a truncated nonsignaling variant of Trop-2, and (ii) CCL5. In preferred embodiments, the polynucleotide encodes (i) a truncated nonsignaling variant of HER2, and (ii) CCL5. In preferred embodiments, the polynucleotide encodes (i) a truncated nonsignaling variant of BCMA, and (ii) CCL5.

In preferred embodiments, the polynucleotide as described herein encodes (i) a truncated nonsignaling variant of CD19, (ii) a truncated nonsignaling variant of BCMA, and (iii) CCL5. In preferred embodiments, the polynucleotide encodes (i) a truncated nonsignaling variant of CD19, (ii) a truncated nonsignaling variant of Trop-2, and (iii) CCL5. In preferred embodiments, the polynucleotide encodes(i) a truncated nonsignaling variant of CD19, (ii) a truncated nonsignaling variant of HER2, and (iii) CCL5.

In some embodiments, the tumor cell is a solid tumor cell. In some embodiments, the tumor cell does not express the tumor associated antigen or tumor specific antigen encoded by the polynucleotide. In some embodiments, the tumor cell expresses the tumor associated antigen or tumor specific antigen encoded by the polynucleotide.

In some embodiments, the genetically modified oHSV as described above is further modified to delete a fragment of nucleic acid of the genome of the oHSV, such that the oHSV is attenuated or removed of certain properties undesirable for the purpose of its intended use. In one embodiment, the genetically modified oHSV is deleted of the internal inverted repeats, a fragment encoding a viral gene, or both. In one embodiment, the fragment of nucleic acid of the oHSV deleted is from positions 117005 to 132096 in the P prototype genome of F strain of HSV-1. In one embodiment, the fragment encoding a viral gene is a fragment of nucleic acid encoding γ34.5. In one embodiment, both copies of gene γ34.5 are deleted.

In some embodiments, the genetically modified oHSV as described above is further modified to encode an immunostimulatory agent, an immunotherapeutic agent, or both. In one embodiment, the immunostimulatory agent is selected from a group consisting of GM-CSF, IL-2, IL-12, IL-15, IL-24, and IL-27. In one embodiment, the immunotherapeutic agent is an anti-PD-1 antibody, an anti-CTLA4 antibody, or an antigen binding fragment thereof. In one embodiment, the genetically modified oHSV encodes IL-12. In one embodiment, the genetically modified oHSV encodes anti-PD-1 antibody or an antigen binding fragment thereof. In one embodiment, the genetically modified oHSV encodes both IL-12 and anti-PD-1 antibody or an antigen binding fragment thereof.

It should be noted that it is required the expression of a truncated nonsignaling variant(s) of a tumor associated/specific antigen and the chemokine is under the control of an immediate-early promoter of HSV, for example, IE4/5 promoter, such that the tumor associated/specific antigen is expressed shortly after the infection of the virus and before the tumor cell lysis by the replication of the virus. The polynucleotide encoding the truncated nonsignaling variant and the polynucleotide encoding the chemokine may be operably linked to a same immediate-early promoter. In another embodiment, the polynucleotide encoding the truncated nonsignaling variant and the polynucleotide encoding the chemokine may be operably linked to different immediate-early promoters. When the oHSV is further armed with immunostimulatory agent, an immunotherapeutic agent, or both, such as IL-12 and anti-PD-1 antibody, the expression of the immunostimulatory agent and/or the immunotherapeutic agent is not necessarily under the control of an immediate-early promoter but preferably under a different and relatively late promoter, such as a CMV promoter or an Egr-1 promoter. In an embodiment, the polynucleotide encoding IL-12 is operably linked to an Egr-1 promoter. In another embodiment, the polynucleotide encoding a scFv-anti-hPD1 is operably linked to a CMV promoter.

In one embodiment, the genetically modified oHSV is incorporated into its genome with a first polynucleotide that encodes a truncated nonsignaling CD19, and a second polynucleotide that encodes CCL5, wherein the expression of the truncated nonsignaling CD19 and the CCL5 is under the control of HSV-1 IE4/5 promoter.

In one embodiment, the genetically modified oHSV is incorporated into its genome with a first polynucleotide that encodes a truncated nonsignaling BCMA, and a second polynucleotide that encodes CCL5, wherein the expression of the truncated nonsignaling BCMA and the CCL5 is under the control of HSV-1 IE4/5 promoter.

In one embodiment, the genetically modified oHSV is incorporated into its genome with a first polynucleotide that encodes a truncated nonsignaling Trop-2, and a second polynucleotide that encodes CCL5, wherein the expression of the truncated nonsignaling Trop-2 and the CCL5 is under the control of HSV-1 IE4/5 promoter.

In one embodiment, the genetically modified oHSV is incorporated into its genome with a first polynucleotide that encodes a truncated nonsignaling HER2, and a second polynucleotide that encodes CCL5, wherein the expression of the truncated nonsignaling HER2 and the CCL5 is under the control of HSV-1 IE4/5 promoter.

In one embodiment, the genetically modified oHSV is incorporated into its genome with a first polynucleotide that encodes a truncated nonsignaling CD19, a second polynucleotide that encodes a truncated nonsignaling BCMA, and a third polynucleotide that encodes CCL5, wherein the expression of the truncated nonsignaling CD19, the truncated nonsignaling BCMA, and the CCL5 is under the control of HSV-1 IE4/5 promoter.

In one embodiment, the genetically modified oHSV is incorporated into its genome with a first polynucleotide that encodes a truncated nonsignaling CD19, a second polynucleotide that encodes a truncated nonsignaling Trop-2, and a third polynucleotide that encodes CCL5, wherein the expression of the truncated nonsignaling CD19, the truncated nonsignaling Trop-2, and the CCL5 is under the control of HSV-1 IE4/5 promoter.

In one embodiment, the genetically modified oHSV is incorporated into its genome with a first polynucleotide that encodes a truncated nonsignaling CD19, a second polynucleotide that encodes a truncated nonsignaling HER2, and a third polynucleotide that encodes CCL5, wherein the expression of the truncated nonsignaling CD19, the truncated nonsignaling HER2, and the CCL5 is under the control of HSV-1 IE4/5 promoter.

In one embodiment, the genetically modified oHSV is incorporated into its genome with a first polynucleotide that encodes a truncated nonsignaling variant of any one selected from a group consisting of CD19, BCMA, Trop-2 and HER2, a second polynucleotide that encodes CCL5, a third polynucleotide that encodes an anti-PD-1 antibody, and a fourth polynucleotide that encodes IL-12, wherein the expression of the truncated nonsignaling CD19 and the CCL5 is under the control of HSV-1 IE4/5 promoter, and wherein the internal inverted repeat of the oHSV is deleted.

In one embodiment, the genetically modified oHSV is incorporated into its genome with a first polynucleotide that encodes a truncated nonsignaling CD19, a second polynucleotide that encodes a truncated nonsignaling variant of any one selected from a group consisting of BCMA, Trop-2 and HER2, a third polynucleotide that encodes CCL5, a fourth polynucleotide that encodes an anti-PD-1 antibody, and a fifth polynucleotide that encodes IL-12, wherein the expression of the truncated nonsignaling CD19 and the CCL5 is under the control of HSV-1 IE4/5 promoter.

In one embodiment, the genetically modified oHSV is incorporated into its genome with a first polynucleotide that encodes a truncated nonsignaling CD19, a second polynucleotide that encodes a truncated nonsignaling variant of any one selected from a group consisting of BCMA, Trop-2 and HER2, a third polynucleotide that encodes CCL5, a fourth polynucleotide that encodes an anti-PD-1 antibody, and a fifth polynucleotide that encodes IL-12, wherein the expression of the truncated nonsignaling CD19 and the CCL5 is under the control of HSV-1 IE4/5 promoter, and wherein the internal inverted repeat of the oHSV is deleted.

In one embodiment, the genetically modified oHSV is incorporated into its genome with a first polynucleotide that encodes a truncated nonsignaling CD19, a second polynucleotide that encodes a truncated nonsignaling variant of any one selected from a group consisting of BCMA, Trop-2 and HER2, a third polynucleotide that encodes CCL5, a fourth polynucleotide that encodes an anti-PD-1 antibody, and a fifth polynucleotide that encodes IL-12, wherein the expression of the truncated nonsignaling CD19 and the CCL5 is under the control of HSV-1 IE4/5 promoter, wherein the internal inverted repeat of the oHSV is deleted, and wherein all single-copy genes of the oHSV are retained.

In one embodiment, the genetically modified oHSV is incorporated into its genome with a first polynucleotide that encodes a truncated nonsignaling CD19, a second polynucleotide that encodes a truncated nonsignaling variant of any one selected from a group consisting of BCMA, Trop-2 and HER2, a third polynucleotide that encodes CCL5, a fourth polynucleotide that encodes an anti-PD-1 antibody, and a fifth polynucleotide that encodes IL-12, wherein the expression of the truncated nonsignaling CD19 and the CCL5 is under the control of HSV-1 IE4/5 promoter, wherein the internal inverted repeat of the oHSV and both copies of γ34.5 are deleted, and wherein all single-copy genes of the oHSV are retained.

In one embodiment, a Poly A tail is located downstream the polynucleotide encoding the truncated antigen and the chemokine. For example, the polynucleotides encoding the truncated non-signaling variants and the chemokine are arranged as 5′-CD19-CCL5-PolyA-3′, 5′-BCMA-CCL5-PolyA-3′, 5′-HER2-CCL5-PolyA-3′, 5′-CD19-BCMA-CCL5-PolyA-3′, 5′-CD19-Trop-2-CCL5-PolyA-3′, or 5′-CD19-HER2-CCL5-PolyA-3′.

In one embodiment, the incorporation of any of the polynucleotides into the genome of the oHSV that encode the truncated nonsignaling variants, the immunotherapeutic agent, and the chemokine does not disrupt the function of the viral genes. For example, the polynucleotide encoding the anti-PD-1 antibody or an antigen binding fragment thereof is introduced between the UL3 and UL4 genes of the virus, and the polynucleotides encoding the truncated non-signaling variant(s) and the chemokine(s) are introduced between the UL37 and UL38 genes of the virus. In addition, in one embodiment, the polynucleotide encoding IL2 replaces the internal inverted repeat of the virus genome.

In the present disclosure, the tumor associated/specific antigen encoded by the oHSV may be heterologous or homologous to the tumor cell infected by the oHSV. In one embodiment, the tumor cell expresses a tumor associated/specific antigen that is different from the tumor associated/specific antigen encoded by the oHSV. For example, the tumor cell overexpresses CD22, while the genetically modified oHSV of the present disclosure expresses CD19, HER2, or both. In another embodiment, the tumor cell expresses a tumor associated/specific antigen that is same with the tumor associated/specific antigen encoded by the oHSV. For example, the tumor cell expresses HER2 at a low level, and the genetically modified oHSV of the present disclosure expresses HER2. In another embodiment, the tumor cell is not detected of a known tumor associated/specific antigen.

The tumor cell infected by the genetically modified oHSV of the present disclosure is a blood tumor cell or a solid tumor cell.

Advantageously, the presentation of a nonsignaling tumor associated/specific antigen on a tumor cell surface converts the tumor cell from a negative cell to a positive cell with respect to that particular tumor associated/specific antigen and thus make the tumor responsive to therapies targeting that particular tumor associated/specific antigen or tumor specific antigen. The expression of the heterologous polynucleotides is controlled under an immediate-early gene promotor, such as IE4/5 of HSV-1, such that the translated products are produced at the extremely early stage upon the entry and replication of the oHSV in the tumor cell. For example, an oHSV may be modified such that it expresses a truncated nonsignaling CD19, a transmembrane protein that is specifically expressed on normal and most neoplastic B cells. The truncated nonsignaling CD19 is then presented onto a tumor cell surface before cell lysis by the infection of the oHSV and servers as a target for a CD19-directed CAR-T therapies, such as Kymriah®, Yescarta®, or Tecartus®. That is, the expression of the nonsignaling CD19 converts a tumor cell that is normally not susceptible to a CD19-directed CAR-T therapy, due to the lack of CD19 antigen on the tumor cell, to a CD19-directed CAR-T susceptible tumor cell. In this case, the tumor would be responsive to a CD19-directed CAR-T therapy.

A key advantage of the genetically modified oHSV that encodes more than one nonsignaling tumor associated/specific antigens resides in that it provides a versatile tool for combinational use with different tumor antigen targeting therapies without the need of repeated designs, tests and manufacturing of an oHSV for each tumor antigen targeting therapy. It showed that when two different nonsignaling tumor associated/specific antigens are encoded by the same oHSV, they can be successfully expressed and presented onto the tumor cell surface at the same time before the tumor cell is lysed due to the infection of the virus. The presentation of two or more different nonsignaling tumor associated/specific antigens would convert the tumor cell into a dual-, or even triple-, positive tumor cell, enabling different tumor-targeting therapies to be effective to the tumor cells. This would aid improving the specificity and efficacy of corresponding tumor targeting therapies, such as a CAR-T cell therapy.

A further advantage of some of the genetically modified oHSV disclosed herein resides in that, in addition to the tumor associated/specific antigens, it encodes at least one chemokine, the expression and release of which further helps trafficking and infiltration of an immune cell towards the tumor cell. It is thus particularly advantageous when a CAR-T, CAR-NK, or the like is used in combination with the oHSV described herein. However, without bound to any theory, the genetically modified oHSV disclosed herein can be used independently and the secretion of the chemokine would induce the chemotaxis of body's immune cells, such as T cells, towards the tumor, and kill the tumor cells together with the anti-tumor effect of the virus.

Combination of oHSV and Tumor Targeting Therapies

Another aspect of the disclosure is directed to the combination of any of the genetically modified oHSV as described above, with a tumor-targeting therapeutic agent, for the treatment of various cancers. As described above, the genetically modified oHSV disclosed herein expresses a nonsignaling tumor associated/specific antigen and then presents the antigen onto the surface of the tumor cell. This provides an opportunity for a therapeutic agent designed to target the tumor associated/specific antigen to target the tumor cell infected by the oHSV.

In the present disclosure, the tumor-targeting therapeutic agent has a targeting moiety specific to the truncated nonsignaling variant of the at least one tumor associated/specific antigen encoded by the oHSV, and an effector moiety for killing or inhibiting the proliferation of a cell of the cancer. The targeting moiety has a specificity to the nonsignaling tumor associated/specific antigen expressed on the tumor cell surface upon entry and replication of the oHSV into the tumor cell. For example, the targeting moiety is an antigen binding domain of an antibody against the tumor associated/specific antigen, such as an antibody, a scFv, a Fab, or the chimeric antigen receptor moiety of a CAR-T cell. The effector moiety is useful for killing or inhibiting the proliferation of a cell of the cancer. For example, the effector moiety is an immune cell including T and nature killer cells, a part of the BiTE that is engageable with a T cell, or the drug moiety of an antibody-drug conjugate.

In some embodiments, the tumor-targeting therapeutic agent is selected from a group consisting of a chimeric antigen receptor T (CAR-T) cell, a chimeric antigen receptor NK (CAR-NK) cell, a bispecific T cell engager (BiTE) and an antibody drug conjugate (ADC). In some embodiments, the tumor-targeting therapeutic agent is a CAR-T cell. In some embodiments, the tumor-targeting therapeutic agent is a CAR-NK cell. In some embodiments, the tumor-targeting therapeutic agent is a BiTE. In some embodiments, the tumor-targeting therapeutic agent is an ADC.

In some embodiments, the tumor-targeting therapeutic agent is a CD19-targeted CAR-T cell. In some embodiments, the tumor-targeting therapeutic agent is a CD19- or EpCAM-targeted BiTE. In some embodiments, the tumor-targeting therapeutic agent is a HER2-, Trop-2-, Nectin-4-, BCMA-, CD33-, CD30-, CD22-, or CD79b-targeted ADC.

In some embodiments, the tumor-targeting therapeutic agent is a CD19-targeted CAR-T cell. In some embodiments, the tumor-targeting therapeutic agent is selected from a group consisting of Tecartus®, Kymriah®, Yescarta®, JWCAR-029, IM19CAR-T, CNCT19, BZ019, HD CD19 CAR-T, pCAR-19B, CD19-CART, CT032, iPD1 CD19 eCAR-T, LCAR-B38M, CT103A, CAR-BCMA T, AU-101, 4SCAR-PSMA, PSMA-CART, P-PSMA-101, C-CAR066, MB-CART20.1, PBCAR20A, LB1095, LB1901, PRGN-3006, AMG553, CT041, CD30.CAR-T, and CAR-GPC3 T.

In some embodiments, the tumor-targeting therapeutic agent is a CD19- or EpCAM-targeted BiTE. In some embodiments, the tumor-targeting therapeutic agent is selected from a group consisting of Blincyto®, AMG420, PF-3135, and GBR1302.

In some embodiments, the tumor-targeting therapeutic agent is a HER2-, Trop-2-, Nectin-4-, BCMA-, CD33-, CD30-, CD22-, or CD79b-targeted ADC. In some embodiments, the tumor-targeting therapeutic agent is selected from a group consisting of Kadcyla®, Enhertu®, SHR-A1811, TAA013, RC-48, BAT8001, ARX788, A166, Trodelvy®, BAT8003, DAC-002, DS-1062, SKB264, RC-108, TR1801-ADC, Padccv®, Polivy®, Adcetris®, Mylotarg®, Blenrep®, PSMA ADC, ADCT-402, PTK7-ADC, and TRS005.

In preferable embodiment, the genetically modified oHSV for combinational use with any of the tumor-targeting therapeutic agents as described above is a genetically modified oHSV in which the genome of the oHSV is incorporated with a polynucleotide encoding (a) truncated nonsignaling variants of two tumor associated/specific antigens, and (b) a chemokine, wherein the expression of the truncated nonsignaling variants and the chemokine is under the control of an immediate-early gene promoter of HSV. In some embodiments, the two tumor associated/specific antigens comprise two tumor associated antigens, which are same or different. In some embodiments, the two tumor associated/specific antigens comprise two tumor specific antigens, which are same or different. In some embodiments, the two tumor associated/specific antigens include one tumor associated antigen and one tumor specific antigen.

In preferable embodiment, the genetically modified oHSV for combinational use with a tumor-targeting therapeutic agent is a genetically modified oHSV expressing truncated nonsignaling variants of both CD19 and BCMA, and CCL5, and the tumor-targeting therapeutic agent is a CD19-targeted CAR-T cell, such as Kymriah®, Yescarta®, or Tecartus®, a CD19-targeted BiTE, such as Blinatumomab, a BCMA-targeted ADC, such as Blenrep®, or any combination thereof.

In preferable embodiment, the genetically modified oHSV for combinational use with a tumor-targeting therapeutic agent is a genetically modified oHSV expressing truncated nonsignaling variants of both CD19 and HER2, and CCL5, and the tumor-targeting therapeutic agent is a CD19-targeted CAR-T cell, such as Kymriah®, Yescarta®, or Tecartus®, a CD19-targeted BiTE, such as Blinatumomab, a HER2-targeted ADC, such as Kadcyla® or Enhertu®, or any combination thereof.

In preferable embodiment, the genetically modified oHSV for combinational use with a tumor-targeting therapeutic agent is a genetically modified oHSV expressing truncated nonsignaling variants of both CD19 and Trop-2, and CCL5, and the tumor-targeting therapeutic agent is a CD19-targeted CAR-T cell, such as Kymriah®, Yescarta®, or Tecartus®, a CD19-targeted BiTE, such as Blinatumomab, a Trop-2-targeted ADC, such as Trodelvy®, or any combination thereof.

The combination of the oHSV and the tumor-targeting therapies can be embodied, for example, as a pharmaceutical kit. Thus, in one aspect, a pharmaceutical kit for treatment of a cancer is provided, which comprises, separately, a genetically modified oncolytic herpes simplex virus (oHSV) as described herein and a tumor-targeting therapeutic agent, wherein the tumor-targeting therapeutic agent has a targeting moiety specific to the truncated nonsignaling variant of the at least one tumor associated/specific antigen encoded by the polynucleotide, and an effector moiety for killing or inhibiting the proliferation of a cell of the cancer.

In some embodiments, the pharmaceutical kit for treatment of a cancer comprises, separately, a genetically modified oncolytic herpes simplex virus (oHSV) encoding (i) a truncated nonsignaling variant of CD19, (ii) a truncated nonsignaling variant of BCMA, and (iii) CCL5; and a CD19- or BCMA-targeted CAR-T, ADC or BiTE. In some embodiments, the CD19- or BCMA-targeted CAR-T, ADC or BiTE is selected from a group consisting of Tecartus®, Kymriah®, Yescarta®, ADCT-402 (ADC Therapeutics), Blinatumomab, JNJ-68284528 (JNJ-4528, Legend Biotech), Blenrep (or GSK2857916), AMG420 (Amgen), and PF-3135 (Pfizer).

In some embodiments, the pharmaceutical kit for treatment of a cancer, comprises, separately, a genetically modified oncolytic herpes simplex virus (oHSV) encoding (i) a truncated nonsignaling variant of CD19, (ii) a truncated nonsignaling variant of Trop-2, and (iii) CCL5; and a CD19- or Trop-2-targeted CAR-T, ADC or BiTE. In some embodiments, the CD19- or Trop-2-targeted CAR-T, ADC or BiTE is selected from a group consisting of Tecartus®, Kymriah®, Yescarta®, ADCT-402 (ADC Therapeutics), Blinatumomab, and Trodelvy® (Immunomedics).

In some embodiments, the pharmaceutical kit for treatment of a cancer, comprises, separately, a genetically modified oncolytic herpes simplex virus (oHSV) encoding (i) a truncated nonsignaling variant of CD19, (ii) a truncated nonsignaling variant of HER2, and (iii) CCL5; and a CD19- or HER2-targeted CAR-T, ADC or BiTE. In some embodiments, the CD19- or HER2-targeted CAR-T, ADC or BiTE is selected from a group consisting of Tecartus®, Kymriah®, Yescarta®, ADCT-402 (ADC Therapeutics), Blinatumomab, AU-101 (Aurora Biopharma), Kadcyla® (Roche), Enhertu® (Daiichi Sankyo) and GBR1302 (Ichnos Sciences SA).

### Treatment Methods

A further aspect of the disclosure is related to a method for treatment of a cancer in a subject. The method comprises administering to the subject a therapeutically effective amount of a genetically modified oHSV as described herein, and a tumor targeting therapeutics as described herein. The administrations of both the oHSV and the tumor targeting therapeutics is performed concurrently or sequentially.

In one embodiment, the subject is firstly administered with a therapeutically effective amount of a genetically modified oHSV as described herein, then a tumor targeting therapeutics as described herein. In this embodiment, the interval between the administrations is in the range of 0.5-12 hours, for example, 0.5-9 hours, 0.5-8 hours, 0.5-7 hours, 0.5-6 hours, 0.5-5 hours, 0.5-4 hours, 0.5-3 hours, 0.5-2 hours, 0.5-2.5 hours, 0.5-1.5 hours, or 0.5-1 hour. For example, the administration of the oHSV is 0.5, 0.75, 1, 1.5, 2, 2.5, 3, 3.5, 4, 4.5, 5, 5.5, 6, 7, 8, 9, 10, 11, or 12 hours before the administration of the tumor targeting therapeutics.

In some embodiments, the method comprises administering to a subject a therapeutically effective amount of a genetically modified oncolytic herpes simplex virus (oHSV) encoding (i) a truncated nonsignaling variant of CD19, (ii) a truncated nonsignaling variant of BCMA, and (iii) CCL5; and a CD19- or BCMA-targeted CAR-T, ADC or BiTE. In some embodiments, the CD19- or BCMA-targeted CAR-T, ADC or BiTE is selected from a group consisting of Tecartus®, Kymriah®, Yescarta®, ADCT-402 (ADC Therapeutics), Blinatumomab, JNJ-68284528 (JNJ-4528, Legend Biotech), Blenrep (or GSK2857916), AMG420 (Amgen), and PF-3135 (Pfizer).

In some embodiments, the method comprises administering to a subject a therapeutically effective amount of a genetically modified oncolytic herpes simplex virus (oHSV) encoding (i) a truncated nonsignaling variant of CD19, (ii) a truncated nonsignaling variant of Trop-2, and (iii) CCL5; and a CD19- or Trop-2-targeted CAR-T, ADC or BiTE. In some embodiments, the CD19- or Trop-2-targeted CAR-T, ADC or BiTE is selected from a group consisting of Tecartus®, Kymriah®, Yescarta®, ADCT-402 (ADC Therapeutics), Blinatumomab, and Trodelvy® (Immunomedics).

In some embodiments, the method comprises administering to a subject a therapeutically effective amount of a genetically modified oncolytic herpes simplex virus (oHSV) encoding (i) a truncated nonsignaling variant of CD19, (ii) a truncated nonsignaling variant of HER2, and (iii) CCL5; and a CD19- or HER2-targeted CAR-T, ADC or BiTE. In some embodiments, the CD19- or HER2-targeted CAR-T, ADC or BiTE is selected from a group consisting of Tecartus®, Kymriah®, Yescarta®, ADCT-402 (ADC Therapeutics), Blinatumomab, AU-101 (Aurora Biopharma), Kadcyla® (Roche), Enhertu® (Daiichi Sankyo) and GBR1302 (Ichnos Sciences SA).

The combinational use of the oHSV described herein and various tumor antigen directed CAR-T cells, ADCs or BiTEs provides a significantly enhanced anti-tumor effects against various tumors. oHSV directly destroys the barriers and manipulates the tumor microenvironment by direct tumor cell lysis. oHSV armed with payload like chemokine and cytokine further improves T cell trafficking and infiltration towards the tumor mass. Also, the highly tumor-specific antigen (for example CD19, BCMA) delivered by oHSV on solid tumor cell surface also improves specificity and safety of tumor targeting therapies, such as CAR-T therapy, by reducing the on-target off-tumor toxicity.

### Sequences

The amino acid or nucleic acid sequences described in this present disclosure are provided in the Table 1 below.

TABLE 1
The amino acid or nucleic acid sequences described in this present disclosure

| SEQ ID NO.# | Sequences |
|---|---|
| 1 | GGAAGCGGAGAGGGCAGAGGAAGTCTGCTAACATGCGGTGACGTCGAGGAGAATCCT |
| | GGACCT |
| 2 | GAAGATCTAATATTTTTATTGCAACTCCCTG |
| 3 | CTAGCTAGCTTATAAAAGGCGCGTCCCGTGG |
| 4 | GCTCTAGATTGCGACGCCCCGGCTC |
| 5 | CCTTAATTAAGGTTACCACCCTGTAGCCCCGATGT |
| 6 | TCCCATGGATTTAACAAACGGGGGGGTGTCG |
| 7 | GGCCCCCGAGGCCAGCATGACGTTATCT |
| 8 | GAGTAACCGCCCCCCCCCCATGCCACCCTCAC |
| 9 | GTGTTTTACTGCCACTACACCCCCGGGGAAC |
| 10 | TCTAGAGATCAGGGTGTTGATCACCACGGAG |
| 11 | GAATTCGGGCTGCCCGCACATCCGGACAATC |
| 12 | CATATGGGACGGCGTGGGTTGCGGACTTTCTG |
| 13 | TTAATTAATCACGCGCATGCCCGCCACTCGCC |
| 14 | MPPPRLLFFLLFLTPMEVRPEEPLVVKVEEGDNAVLQCLKGTSDGPTQQLTWSRESP |
| | LKPFLKLSLGLPGLGIHMRPLAIWLFIFNVSQQMGGFYLCQPGPPSEKAWQPGWTVN |
| | VEGSGELFRWNVSDLGGLGCGLKNRSSEGPSSPSGKLMSPKLYVWAKDRPEIWEGEP |
| | PCVPPRDSLNQSLSQDLTMAPGSTLWLSCGVPPDSVSRGPLSWTHVHPKGPKSLLSL |
| | ELKDDRPARDMWVMETGLLLPRATAQDAGKYYCHRGNLTMSFHLEITARPVLWHWLL |
| | RTGGWKVSAVTLAYLIFCLCSLVGILHLQRALVLRRKR |
| 15 | MLQMAGQCSQNEYFDSLLHACIPCQLRCSSNTPPLTCQRYCNASVTNSVKGTNAILW |
| | TCLGLSLIISLAVFVLMFLL |
| 16 | MELAALCRWGLLLALLPPGAASTQVCTGTDMKLRLPASPETHLDMLRHLYQGCQVVQ |
| | GNLELTYLPTNASLSFLQDIQEVQGYVLIAHNQVRQVPLQRLRIVRGTQLFEDNYAL |
| | AVLDNGDPLNNTTPVTGASPGGLRELQLRSLTEILKGGVLIQRNPQLCYQDTILWKD |
| | IFHKNNQLALTLIDTNRSRACHPCSPMCKGSRCWGESSEDCQSLTRTVCAGGCARCK |
| | GPLPTDCCHEQCAAGCTGPKHSDCLACLHFNHSGICELHCPALVTYNTDTFESMPNP |
| | EGRYTFGASCVTACPYNYLSTDVGSCTLVCPLHNQEVTAEDGTQRCEKCSKPCARVC |
| | YGLGMEHLREVRAVTSANIQEFAGCKKIFGSLAFLPESFDGDPASNTAPLQPEQLQV |
| | FETLEEITGYLYISAWPDSLPDLSVFQNLQVIRGRILHNGAYSLTLQGLGISWLGLR |
| | SLRELGSGLALIHHNTHLCFVHTVPWDQLFRNPHQALLHTANRPEDECVGEGLACHQ |
| | LCARGHCWGPGPTQCVNCSQFLRGQECVEECRVLQGLPREYVNARHCLPCHPECQPQ |
| | NGSVTCFGPEADQCVACAHYKDPPFCVARCPSGVKPDLSYMPIWKFPDEEGACQPCP |
| | INCTHSCVDLDDKGCPAEQRASPLTSIISAVVGILLVVVLGVVFGILI |
| 17 | MARGPGLAPPPLRLPLLLLVLAAVTGHTAAQDNCTCPTNKMTVCSPDGPGGRCQCRA |
| | LGSGMAVDCSTLTSKCLLLKARMSAPKNARTLVRPSEHALVDNDGLYDPDCDPEGRF |
| | KARQCNQTSVCWCVNSVGVRRTDKGDLSLRCDELVRTHHILIDLRHRPTAGAFNHSD |
| | LDAELRRLFRERYRLHPKFVAAVHYEQPTIQIELRQNTSQKAAGDVDIGDAAYYFER |
| | DIKGESLFQGRGGLDLRVRGEPLQVERTLIYYLDEIPPKFSMKRLTAGLIAVIVVVV |
| | VALVAGMAVLVI |

### EXAMPLES

Constructions of oHSV-1 T7201, T7202, T7203, T7204, T7011, T7012, and T7013

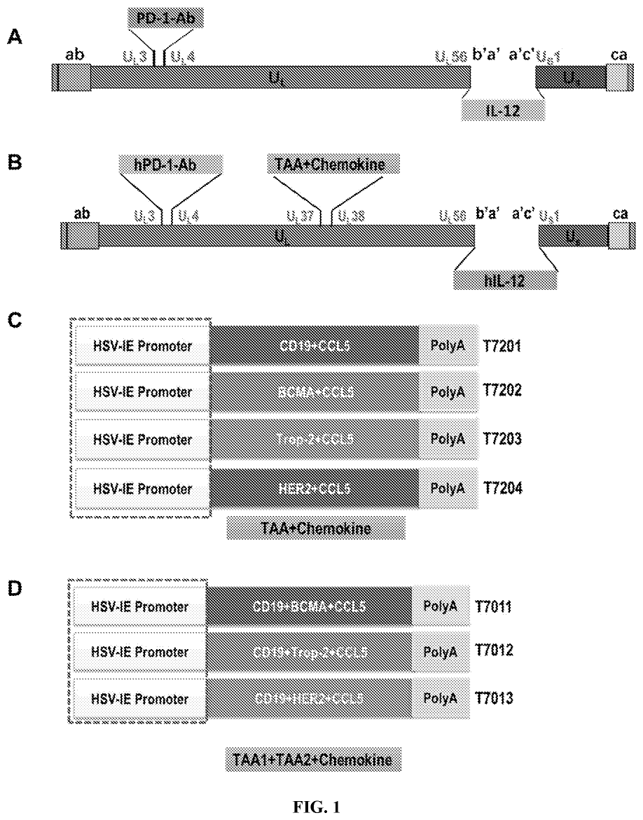

Oncolytic herpes simplex virus (oHSV-1) T7201, T7202, T7203 and T7204 carry coding sequences of IL-12, anti-PD-1 antibody, CCL5, and one truncated nonsignaling variant of tumor associate antigens (TAAs) serving as biomarker. The truncated nonsignaling variant of biomarker expressed by T7201, T7202, T7203 and T7204 are CD19, BCMA, Trop-2, and HER2, respectively. FIG. 1C show schematic diagram of the virus backbones of T7201 to T7204.

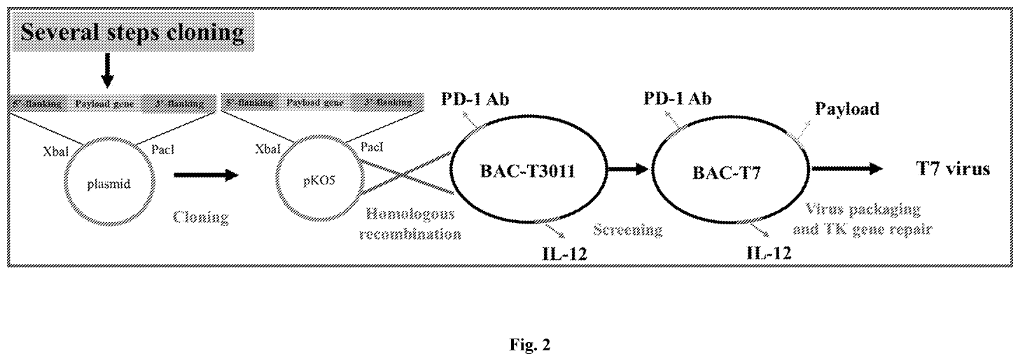

Oncolytic herpes simplex virus (oHSV-1) T7011, T7012, and T7013 carry coding sequences of IL-12, anti-PD-1 antibody, CCL5, and two truncated nonsignaling variant of tumor associate antigens (TAAs) serving as biomarkers. The truncated nonsignaling variant of biomarkers expressed by T7011, T7012, and T7013 are CD19 plus BCMA, CD19 plus Trop-2, and CD19 plus HER2, respectively. FIG. 1D shows schematic diagram of the virus backbones of T7011, T7012, and T7013. FIG. 2 shows the flowchart of construction of the T7 series oHSV.

The two biomarkers and CCL5 coding sequences linked by T2A self-cleaving peptide sequence (SEQ ID NO. 1) are translated in one open reading frame driven by HSV-1 immediate early gene promoter (IE4/5 promoter). The expression cassette is inserted between UL37 and UL38 genes.

In addition, T7201, T7202, T7203, T7204, T7011, T7012, and T7013 comprise an insertion of an anti-human PD-1 antibody expression cassette between UL3 and UL4, and a modified internal repeat (IR) region replaced by an IL-12 expression cassette. The recombinant virus was constructed in several steps with the aid of bacterial artificial chromosome (BAC) system. The details of viral construction are described following.

IL-12 expression cassette is flanked by upstream of nucleotides 117005 and downstream of nucleotides 132096 in the context of a wild type genome which were PCR amplified from HSV-1 viral genome by two sets of primers respectively (SEQ ID Nos:2-3) and (SEQ ID Nos:4-5) and inserted into a gene replacement plasmid pKO5 to generate pKO1407. pKO1407 was then transfected to Escherichia coli with wild-type BAC by electroporation to generate BAC-T2010. Then, cassette of CMV promoter driving the anti-PD-1 antibody gene flanked by upstream of nucleotides 11658 and downstream of nucleotides 11659 in the context of a wild type genome which were PCR amplified from HSV-1 viral genome by two sets of primers respectively (SEQ ID Nos:6-7) and (SEQ ID Nos:8-9) was ligated into pKO5 at the sites of BglII and PacI to generate the pKOE1002 plasmid. pKOE1002 plasmid was then transfected to Escherichia coli harboring BAC-T2010 by electroporation to generate BAC-T3011. The expression cassette which contains one or two biomarkers (i.e., tumor associated/specific antigen) and CCL5 genes was flanked by upstream of nucleotides 84220 and downstream of nucleotides 84221 in the context of a wild type genome. The upstream and downstream flanking sequences were PCR amplified from HSV-1 viral genome by two sets of primers respectively (SEQ ID Nos: 10-11) and (SEQ ID Nos:12-13). The DNA fragment comprises biomarkers, CCL5, and flanking sequences was ligated into pKO5 at the sites of Xbal and PacI to generate the pKO7201, pKO7202, pKO7203, pKO7204, pKO7011, pKO7012, and pKO7013 plasmids. pKO7201, pKO7202, pKO7203, pKO7204, pKO7011, pKO7012, and pKO7013 plasmids were then transfected to Escherichia coli harboring BAC-T3011 by electroporation to generate BAC-T7201, BAC-T7202, BAC-T7203 BAC-T7204, BAC-T7011, BAC-T7012, BAC-T7013, respectively. T7201, T7202, T7203, T7204, T7011, T7012, and T7013 viruses were obtained by transfection of corresponding BAC plasmids following by several steps of plaque purification and amplification in Vero cells.

### Virus Titration

Virus titer was measured by plaque forming assay. Briefly, viral stock was serially diluted and then inoculated monolayer Vero cells in T25 flask. After absorption for 2 hours, the medium was replaced with DMEM medium supplemented with 1% FBS plus 0.05% (wt/vol) human pooled immunoglobulin for 72 hours. The cells were fixed with absolute methanol for 5 minutes, rinsed with distilled water and stained with crystal violet. The plaque was counted to calculate infectious virus particle titer. The titers of T7011, T7012 and T7013 are shown in Table 2 below, and the titers of T7201, T7202, T7203 and T7204 are shown in Table 4.

TABLE 2
T7011, T7012 and T7013 oHSV virus titer

| Virus | Virus Titer |
|---|---|
| T7011 | 5.60 × 10⁷ PFU/mL |
| T7012 | 3.05 × 10⁷ PFU/mL |
| T7013 | 9.25 × 10⁷ PFU/mL |

### CCL5 Secretion Detection Post Virus Infection

Human embryo kidney 293T, human laryngeal cancer Hep-2, and human tongue squamous cancer Tca8113 cells were seeded into T25 flask at a density of 1×10⁶ cells per flask. After overnight incubation, the cells were mock infected or infected at 1 PFU of T7011, per cell. After 2-hour incubation, the inoculum was replaced with fresh culture medium. The cell supernatant was harvested at 0, 4, 8, 12, 24, 48, 72, and 96-hour post infection for ELISA assay to quantify CCL5 secretion. FIG. 3 shows the CCL5 release after T7011 infection in 293T, Hep-2, and Tca8113 cells. The expression and release of CCL5 was fast and strong. Secreted CCL5 was detected as soon as 4 hours post-infection and peaked as high as 5,000 pg/ml. The secretion was stable and retained for up to at least 4 days after T7011 viral infection. It thus showed that the CCL5 secretion.

### Expression of IL-12, Anti-PD-1 Antibody and CCL5 by ELISA Assay

Vero cells were seeded into T150 flask at a density of 6×10⁶ cells per flask. After overnight incubation, the cells were infected at 0.01 PFU of T7201, T7202, T7203, T7204, T7011, T7012 and T7013 per cell. The cell supernatants collected at 48-hour post-infection were used for ELISA assay to detect the expression level of IL-12, anti-PD-1 antibody and CCL5. Results were shown in Table 3 and Table 4. As shown in Table 3, the expression of CCL5 was at a high level and very stable among the viruses tested. The expressions of IL-12 and PD-1 Ab were substantially same between T7011 and T7013, while T7012 showed the highest IL-12 expression and the lowest PD-1 Ab expression.

TABLE 3
The expression level of IL-12, anti-PD-1
antibody and CCL5 post virus infection

| | IL-12 | PD-1 Ab | CCL5 |
|---|---|---|---|
| | concentration | concentration | concentration |
| Virus | (pg/mL) | (pg/mL) | (pg/mL) |
| T7011 | 547 | 2897 | 5453 |
| T7012 | 787 | 1923 | 5394 |
| T7013 | 500 | 3055 | 5496 |

TABLE 4
T7201, T7202, T7203 and T7204 oHSV virus titer and the expression
level of IL-12, anti-PD-1 Fab and CCL5 post virus infection

| | Titer | IL-12 | Anti-PD-1 Fab | CCL5 |
|---|---|---|---|---|
| Virus | (PFU/mL) | (pg/mL) | (pg/mL) | (pg/mL) |
| T7201 | 1.08 × 10⁸ | 731.37 | 749.33 | 9811.34 |
| T7202 | 9.70 × 10⁷ | 544.79 | 671.69 | 42267.47 |
| T7203 | 9.58 × 10⁷ | 541.90 | 882.49 | 44351.20 |
| T7204 | 3.90 × 10⁷ | 387.92 | 356.72 | 33275.46 |

### Expression and Presentation of Truncated CD19, BCMA, Trop-2, HER2 on Cell Surface by Immunofluorescence Assay

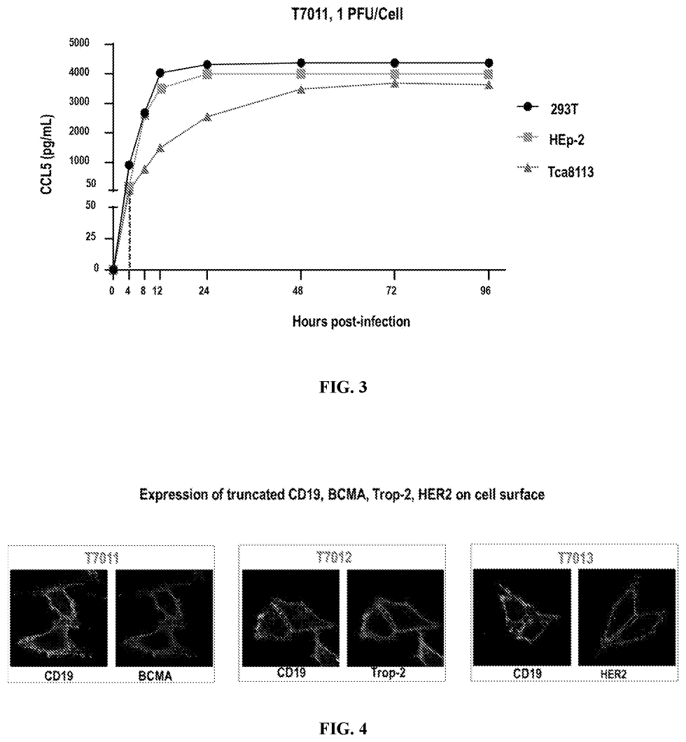

Hep-2 cells (4×10⁵) were seeded in coverslip in individual wells of 6-well plate and incubated for 24 hours to allow cells to adhere. And then the cells were mock infected or exposed separately to 5 PFU of T7011, T7012 and T7013 virus per cell for 1 hour. The inoculum was replaced with fresh culture medium. Cells were rinsed with PBS and fixed with 4% paraformaldehyde for 10 min at room temperature at the indicated times followed by blocking with 5% skim milk. The cells infected by T7011 were co-stained with an antibody to CD19 (Cat. 302204, Biolegend) as well as those to BCMA (Cat. NBP1-97637, Novus); The cells infected by T7012 were co-stained with an antibody to CD19 (Cat. 302204, Biolegend) and those to Trop-2 (Cat. PA5-47030, Invitrogen); The cells infected by T7013 were stained with an antibody to CD19 (Cat. 302204, Biolegend) and primary HER2 antibody (Cat. MAB1129-100, R&D systems) overnight at 4° C., respectively. The cells were then incubated with Alexa Fluor 488-conjugated anti-mouse (Cat. A32766, Invitrogen,), Alexa Fluor 568-conjugated anti-rabbit (Cat. A11036, Invitrogen) and Alexa Fluor 568-conjugated anti-goat (Cat. A11057, Invitrogen) secondary antibody at room temperature for 1 hour. Cells were then washed with PBS and embedded in mounting medium (Cat. 8961S, Cell Signaling Technology). The images were captured and processed using a Nikon confocal laser-scanning microscope (HD25, magnification, 120×) and shown in FIG. 4. As can be seen from FIG. 4, the different truncated antigens encoded by T7011, T7012, and T7013, respectively, were concurrently expressed on tumor cell surfaces.

### Neurovirulence Study

6-week-old female BALB/c mice were anesthetized and then intracranially injected with 50 μL of 10-fold serial dilutions in groups of 8 mice per dilution of HSV-1 (F), T3011, T7011, T7012, or T7013 viruses. The same volume of DPBS containing 10% glycerin was inoculated as mock treatment control group. The mice were monitored for 14 days and the 50% lethal dose (LD50) was calculated from the mortality data according to the Reed and Muench's method.

As shown in Table 5 below, LD₅₀ value of T7011, T7012, T7013 and T3011 is 158-fold, 316-fold, 100-fold and 268-fold higher than HSV-1 (F) respectively, indicating the same as T3011 virus, the neurotoxicity of T7011, T7012 and T7013 are significantly attenuated when compared to HSV-1 (F).

TABLE 5
LD₅₀ value of T7011, T7012, T7013 and T3011

| | | | LD₅₀ Ratio Compared to |
|---|---|---|---|
| Drug | Log₁₀(LD₅₀) | LD₅₀ | HSV-1 (F) |
| T7011 | 4.376 | 2.38 × 10⁴ PFU | 158 |
| T7012 | 4.676 | 4.74 × 10⁴ PFU | 316 |
| T7013 | 4.176 | 1.50 × 10⁴ PFU | 100 |
| T3011 | 4.605 | 4.02 × 10⁴ PFU | 268 |
| HSV-1(F) | 2.176 | 1.50 × 10² PFU | NA |

Anti-Tumor Activity of T7 Series oHSV Virus

Tumor cells were seeded onto 96-well plate at a density of 10000 cells per well. After overnight incubation, the cells in triplicate were infected with T3011, T7011, T7012 and T7013 at 0.01, 0.1, 1, 5, 10, 33.33 and 100 PFU per cell. After 48-hours infection(p.i.), the cell viability was determined by CellTiter-Glo. The cell growth inhibition rate was calculated according to manufacturer's instructions. The concentration (PFU/cell) resulting in 50% of cell growth inhibition (IC₅₀) by viral infection was calculated by fitting data into dose-response curves using GraphPad Prism software.

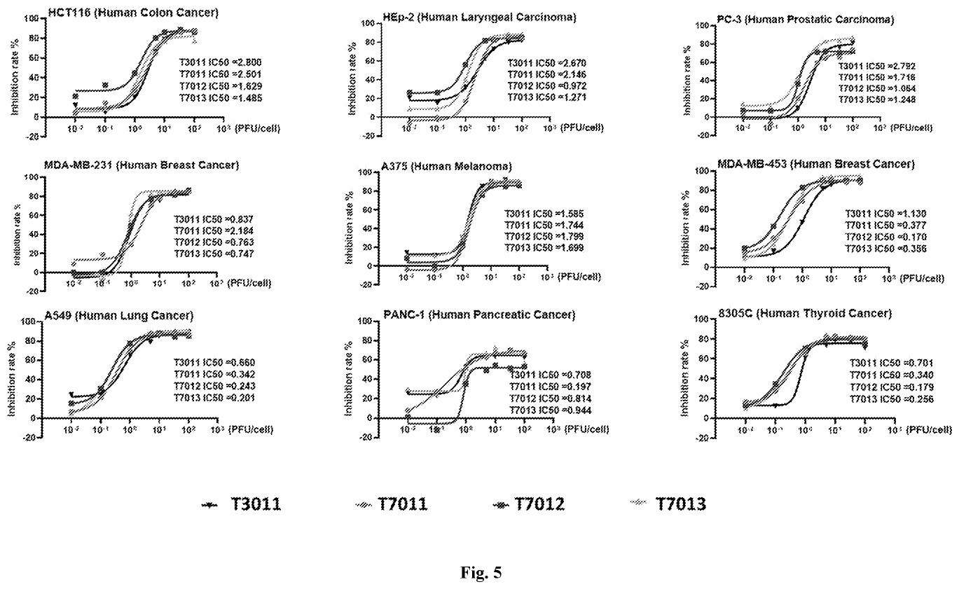

As shown in FIG. 5, the IC₅₀ values of T7 series are comparable to T3011, indicating that T7 serial viruses have similar broad anti-tumor activity compared to T3011. Meanwhile, the IC₅₀ values on HCT116, Hep-2, PC-3, MDA-MB-231, and A375 cells are slightly higher than that in other tumor cell lines, indicating those cell lines are relatively resistant to T7 serial viruses' infection and then selected for the further combo studies.

Infection Activity of T7 Series oHSV Virus

Hep-2 cells, un-transduced normal T cells and CD19 CAR-T (CAR-T) cells were seeded onto 12-well plates at a density of 5×10⁵ cells per well and infected with HSV-1 (F), T7011, T7012, and T7013 at 1 PFU per cell. The cell pellets were harvested at 24 and 48 hours (h) post-infection and then washed by PBS. The cell pellets were then resuspended in DPBS+10% glycerin, and followed by three freeze-thaw cycles. Virus progeny were titrated on Vero cells.

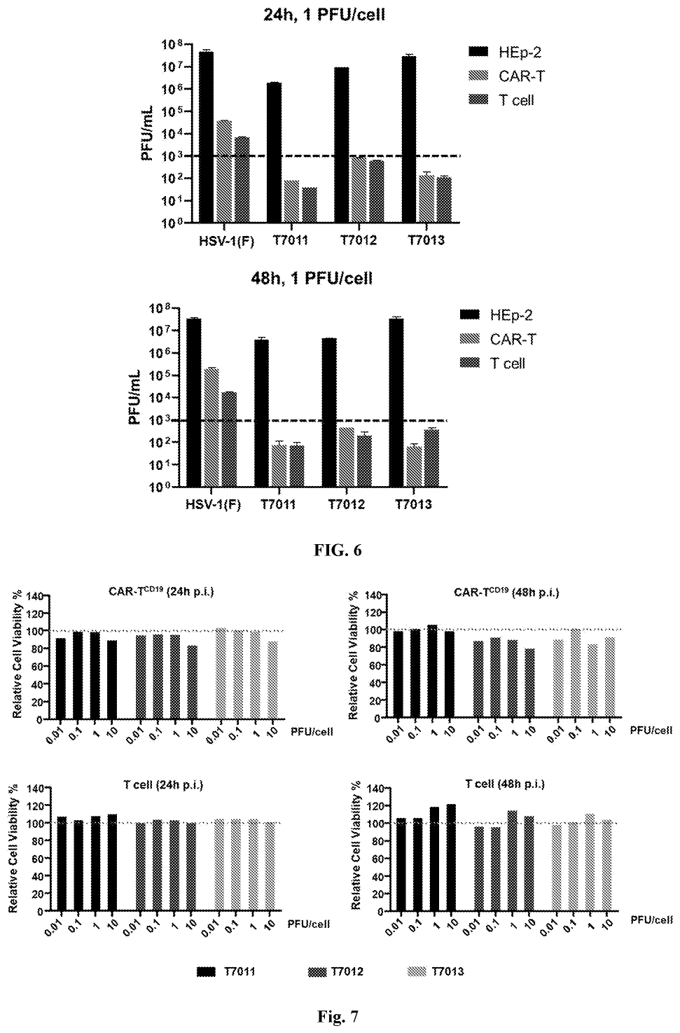

As shown in FIG. 6, the viral yields in all viral infected Hep-2 cells are significantly higher than those in normal T cells and CAR-T cells. Particularly, the yields of T7 serial viruses in both normal T cells and CAR-T cells are no more than 103 PFU/mL at either 24 h or 48 h post-infection. These results indicates that the wild type HSV-1 (F) virus has low infectious activity in CAR-T or normal T cells, while attenuated T7011, T7012 and T7013 viruses have no infection activity.

Cell Killing Activity of T7 Series oHSV Virus

CD19 CAR-T (CAR-TCD19) cells and un-transduced normal T cells were seeded onto 96-well plate at 4×10⁴ cells per well and infected with T7011, T7012 and T7013 at 0.01, 0.1, 1, and 10 PFU per cell in triplicate. The cell viability was determined by CellTiter-Glo at 24 and 48 h post-infection (p.i). The relative cell viability was calculated as the percentage of untreated cells.

As shown in FIG. 7, the cell viability has no reduction upon T7 serial virus infection, proving that T7 serial viruses have no cell killing activity against either CAR-T or normal T cells.

Anti-Tumor Effect of T7011 Combo with CAR-TCD19

Human laryngeal carcinoma cell Hep-2, human melanoma cell A375, and human prostatic carcinoma PC-3 cells were seeded onto 96-well plate at a density of 1×10⁴ cells per well. After overnight incubation, the cells were infected with or without T7011 at a 0.01, 0.03, 0.1, 0.3, and 1 PFU per cell in triplicate wells. At 24-hours post-infection, 4×10⁴ cells per well of CAR-T^{CD19} or T cells at a 4:1 effector to target (E:T) ratio were added for co-culture with tumor cells. Un-transduced normal T cells were served as control. After 24-hours co-culture, the cell viability was determined by CellTiter-Glo. The relative cell viability was calculated as the percentage of untreated cells.

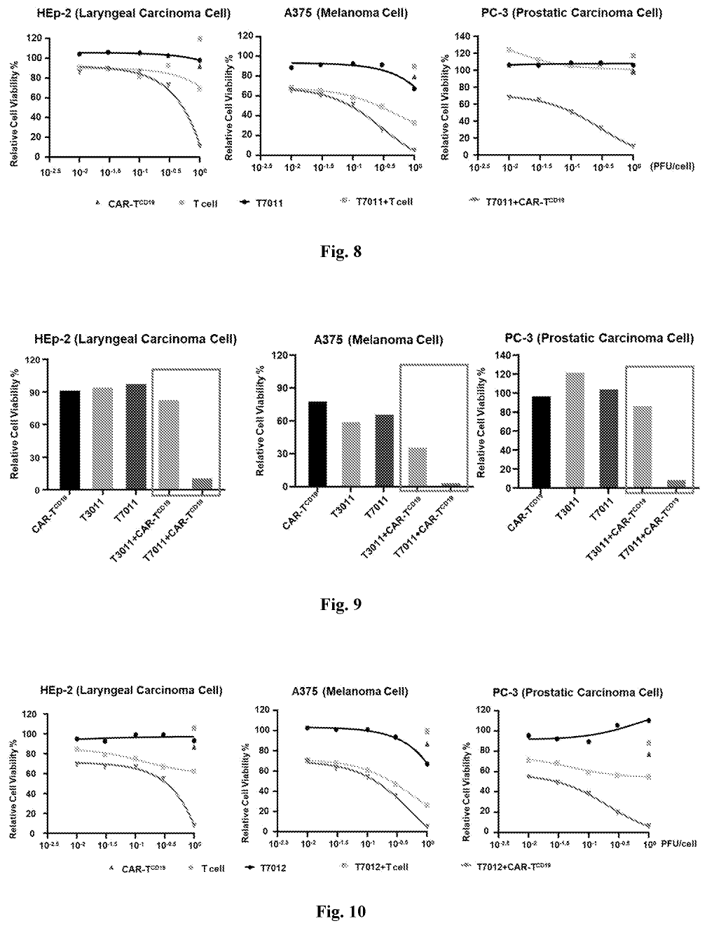

As shown in FIG. 8, combo of T7011 with CAR-TCD19 shows ≥60% higher effect than the single agent. The results demonstrate that, the anti-tumor effect is significantly enhanced in T7011 and CAR-T^{CD19} combo treatment group when compared to T7011 and CAR-T alone group. By contrast, T7011 and normal T cell combo treatment shows a slight anti-tumor effect.

Further, human laryngeal carcinoma cell Hep-2, human melanoma cell A375, and human prostatic carcinoma PC-3 cells were seeded onto 96-well plate at a density of 1×104 cells per well. After overnight incubation, the cells were infected with or without T7011 or T3011 at 1 PFU per cell in triplicate. At 24-hours post-infection, 4×10⁴ cells per well of CAR-T^{CD19} cells were added at a 4:1 effector to target (E:T) ratio for another 24-hours co-culture. Un-treated cell served as untreated control. The cell viability was determined by CellTiter-Glo. The relative cell viability was calculated as the percentage of untreated cells.

As shown in FIG. 9, only T7011 and CAR-T^{CD19} combo treatment has a significant reduction of cell viability compared to single treatment group as well as T3011 and CAR-T combo treatment group. As control, T3011 combo with CAR-TCD19 has no effect. All these results indicates that T7011 virus infection can specifically synergize CAR-TCD19 anti-tumor activity.

Anti-Tumor Effect of T7012 Combo with CAR-T^{CD19}

Human laryngeal carcinoma cell Hep-2, human melanoma cell A375, and human prostatic carcinoma PC-3 cells were seeded onto 96-well plate at a density of 1×104 cells per well. After overnight incubation, the cells were infected or uninfected with T7012 at a 0.01, 0.03, 0.1, 0.3, and 1 PFU per cell in triplicate wells. At 24-hours post-infection, 4×104 cells per well of CAR-T^{CD19} or T cells at a 4:1 effector to target (E:T) ratio were added for co-culture with tumor cells. Un-transduced normal T cells were served as control. After 24-hours co-culture, the cell viability was determined by CellTiter-Glo. The relative cell viability was calculated as the percentage of untreated cells.

As shown in FIG. 10, combo of T7012 with CAR-T^{CD19} shows ≥50% higher effect than the single agent. The results demonstrate that, the anti-tumor effect is significantly enhanced in T7012 and CAR-T^{CD19} combo group compared to T7012 and CAR-T alone group. By contrast, T7012 and normal T cell combo showed a slight anti-tumor effect.

Further, human laryngeal carcinoma cell Hep-2, human melanoma cell A375, and human prostatic carcinoma PC-3 cells were seeded onto 96-well plate at a density of 1×10+ cells per well. After overnight incubation, the cells were infected with or without T7012 or T3011 at 1 PFU per cell in triplicate. At 24 hours post-infection, 4×10⁴ cells per well of CAR-T^{CD19} cells were added at a 4:1 effector to target (E:T) ratio for another 24 hours co-culture. Un-treated cell served as untreated control. The cell viability was determined by CellTiter-Glo. The relative cell viability was calculated as the percentage of untreated cells.

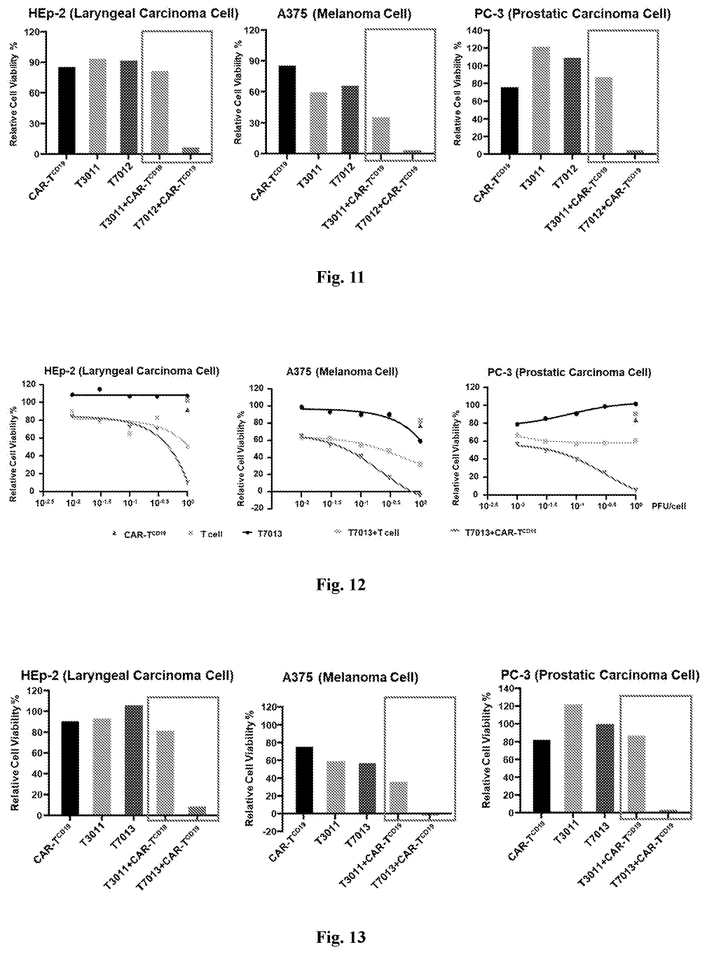

As shown in FIG. 11, only T7012 and CAR-T^{CD19} combo treatment has a significant reduction of cell viability compared to single treatment group as well as T3011 and CAR-T combo treatment group. As control, T3011 combo with CAR-T^{CD19} has no effect. All these results indicates that T7012 virus infection can specifically synergize CAR-T^{CD19} anti-tumor activity.

Anti-Tumor Effect of T7013 Combo with CAR-T^{CD19}

Human laryngeal carcinoma cell Hep-2, human melanoma cell A375, and human prostatic carcinoma PC-3 cells were seeded onto 96-well plate at a density of 1×10⁴ cells per well. After overnight incubation, the cells were infected or uninfected with T7013 at a 0.01, 0.03, 0.1, 0.3, and 1 PFU per cell in triplicate wells. At 24-hours post-infection, 4×10⁴ cells per well of CAR-T^{CD19} or T cells at a 4:1 effector to target (E:T) ratio were added for co-culture with tumor cells. Un-transduced normal T cells were served as control. After 24-hours co-culture, the cell viability was determined by CellTiter-Glo. The relative cell viability was calculated as the percentage of untreated cells.

As shown in FIG. 12, combo of T7013 with CAR-T^{CD19} showed ≥60% higher effect than the single agent. The results demonstrate that, the anti-tumor effect is significantly enhanced in T7013 and CAR-T^{CD19} combo group compared to T7013 and CAR-T alone group. By contrast, T7013 and normal T cell combo showed a slight anti-tumor effect.

Further, Human laryngeal carcinoma cell Hep-2, human melanoma cell A375, and human prostatic carcinoma PC-3 cells were seeded onto 96-well plate at a density of 1×10⁴ cells per well. After overnight incubation, the cells were infected with or without T7013 or T3011 at 1 PFU per cell in triplicate. At 24 hours post-infection, 4×10⁴ cells per well of CAR-T^{CD19} cells were added at a 4:1 effector to target (E:T) ratio for another 24-hours co-culture. Un-treated cell served as untreated control. The cell viability was determined by CellTiter-Glo. The relative cell viability was calculated as the percentage of untreated cells.

As shown in FIG. 13, only T7013 and CAR-T^{CD19} combo treatment has a significant reduction of cell viability compared to single treatment group as well as T3011 and CAR-T combo treatment group. As control, T3011 combo with CAR-T^{CD19} has no effect. All these results indicates that T7013 virus infection can specific synergistic CAR-T^{CD19} anti-tumor activity.

Cell Killing Activity of T7 Series (T7011, T7012, and T7013) oHSV Virus in CAR-NK^{CD19} and NK Cells

NK cells isolated from PBMC according to methods known in the art. CD19 CAR-NK (CAR-NK^{CD19}) cells and un-transduced normal NK cells were seeded onto 96-well plate at 4×10⁴ cells per well and infected with HSV-1(F), T7011, T7012 and T7013 at 0.01, 0.1, 1, and 10 PFU per cell in triplicate. The cell viability was determined by CellTiter-Glo at 24, 48 h and 72 h post-infection (p.i). The relative cell viability was calculated as the percentage of untreated cells.

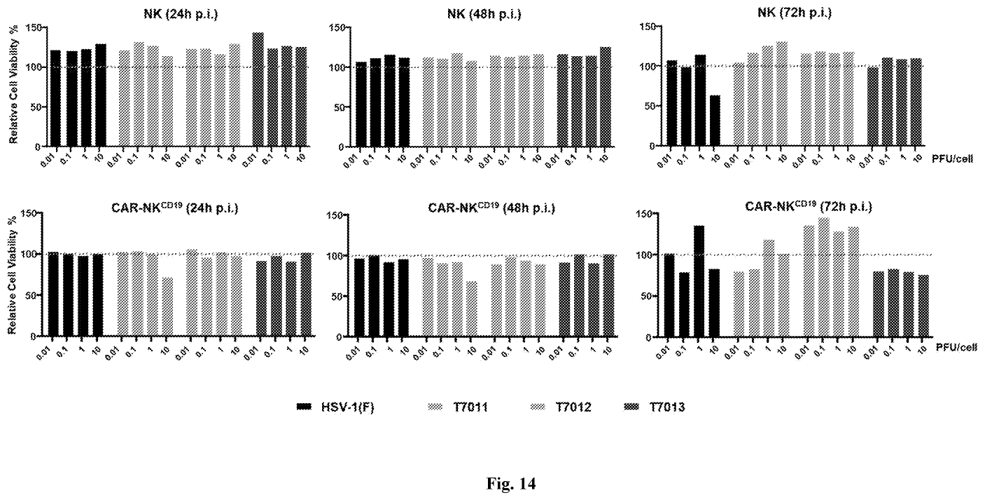

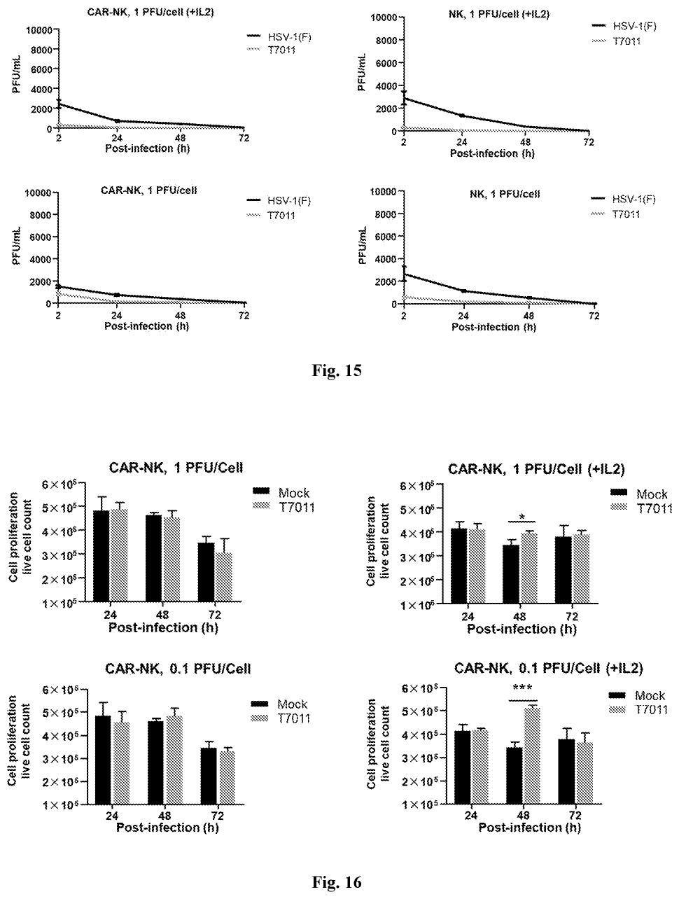

As shown in FIG. 14, the cell viability has no reduction upon T7 serial virus infection, proving that T7 serial viruses have no cell killing activity against either CAR-NK or normal NK cells.

### Viral Replication of HSV-1 (F) and T7011 in CAR-NK^{CD19} and NK Cells

CAR-NK^{CD19} and NK cells were seeded in 12-well plates at a density of 5×10⁵ cells/well were infected with HSV-1 (F) and T7011 at 1 PFU per cell in the presence of IL-2 (+IL-2) or absence of IL-2. The cell pellets were harvested at 2, 24, 48 and 72 hour (h) post-infection. The cell pellets were then washed by PBS and followed by resuspension in DPBS+10% glycerin, freeze-thaw 3 times. Virus progeny were titrated on Vero cells.

As shown in FIG. 15, T7011 virus have no infection activity in CAR-NK^{CD19} or normal NK cells.

### Effects of T7011 on the Proliferation of CAR-NK^{CD19} Cells

CAR-NK^{CD19} cells seeded in 12-well plates at a density of 5×10⁵ cells/well were infected with or without HSV-1 (F) and T7011 at 0.1 or 1 PFU per cell in the presence (+IL-2) or absence of IL-2. The cell pellets were harvested at 24, 48 and 72 hour (h) post-infection and the live cells were determined with trypan blue staining.

As shown in FIG. 16, T7011 has no negative effect on CAR-NK^{CD19} cell proliferation.

### Effects of T7011 on the Proliferation of CAR-NK Cells

NK cells seeded in 12-well plates at a density of 5×10⁵ cells/well were infected with or without HSV-1 (F) and T7011 at 0.1 or 1 PFU per cell in the presence (+IL-2) or absence of IL-2. The cell pellets were harvested at 24, 48 and 72 hour (h) post-infection and the live cells were determined with trypan blue staining.

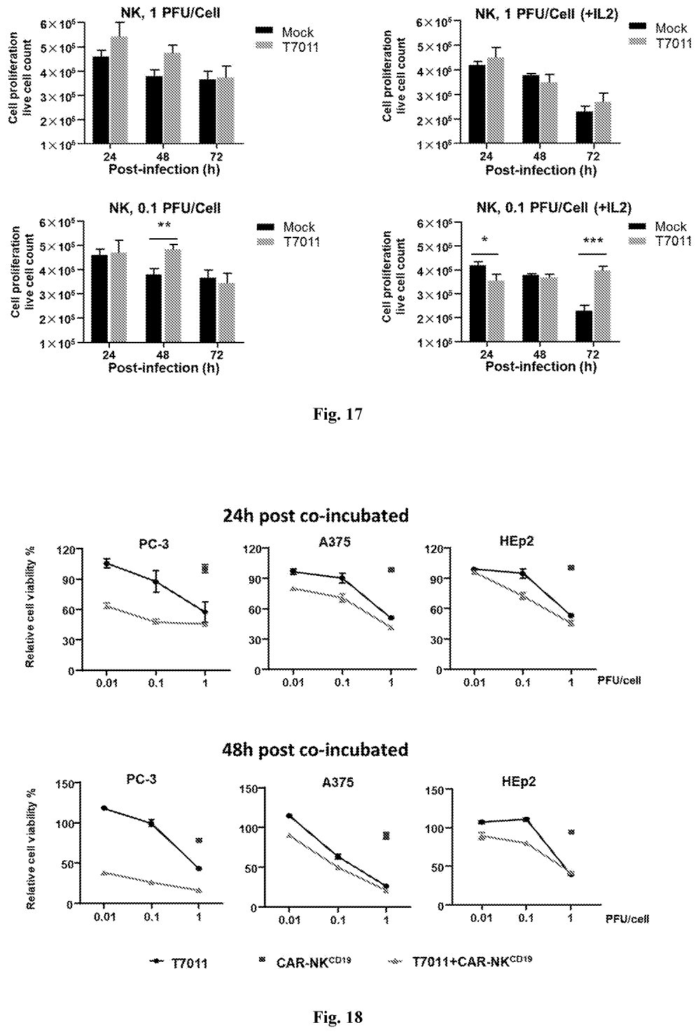

As shown in FIG. 17, T7011 has no negative effect on NK cell proliferation.

Cell Killing Effect of T7011 Combo with CAR-NK^{CD19}

Human laryngeal carcinoma cell Hep-2, human melanoma cell A375, and human prostatic carcinoma PC-3 cells were seeded onto 96-well plate. After overnight incubation, the cells were infected with T7011 at a 0.01, 0.1 and 1 PFU per cell in triplicate wells. At 24-hours post-infection, CAR-NK^{CD19} cells at a 2:1 effector to target (E:T) ratio were added for co-culture with tumor cells. After another 24-hours or 48-hours, the cell viability was determined by CellTiter-Glo. The relative cell viability was calculated as the percentage of untreated cells.

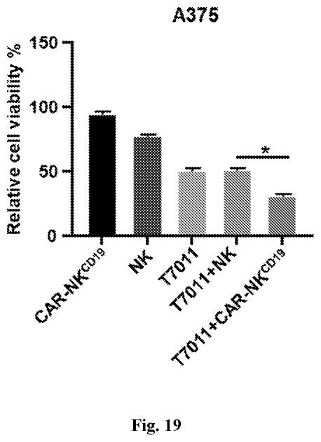

As shown in FIG. 18, combo of T7011 with CAR-NK^{CD19} showed higher effect than the single agent.

Further, human melanoma cell A375 cells were seeded onto 96-well plate at a density of 1×10⁴ cells per well. After overnight incubation, the cells were infected with NK, CAR-NK^{CD19} or T7011 at 1 PFU per cell in triplicate. At 24 hours post-infection, CAR-NK^{CD19} or NK cells were added to T7011-infected cells at a 2:1 effector to target (E:T) ratio for another 24-hours co-culture. The cell viability was determined by CellTiter-Glo. The relative cell viability was calculated as the percentage of untreated cells.

As shown in FIG. 19, T7011 and CAR-NK^{CD19} combo treatment has a significant reduction of cell viability compared to single treatment group as well as T7011 and NK combo treatment group. All these results indicates that T7011 virus infection can specific synergistic CAR-NK^{CD19} anti-tumor activity.

1. A genetically modified oncolytic herpes simplex virus (oHSV),
wherein the genome of the oHSV is incorporated with a polynucleotide encoding
(a) a truncated nonsignaling variant of at least one tumor associated/specific antigen, and
(b) at least one chemokine,
wherein the expression of the truncated nonsignaling variant and the at least one chemokine is under the control of an immediate-early gene promoter of HSV, and
wherein the truncated nonsignaling variant is expressed and presented on a tumor cell surface as a biomarker upon replication of the oHSV in the tumor cell, and the at least one chemokine is expressed and released to induce chemotaxis of an immune cell towards the tumor cell.

2. The genetically modified oHSV of claim 1, wherein the at least one tumor associated/specific antigen is selected from a group consisting of HER2, PSMA, BCMA, CD20, CD33, CD19, CD22, CD123, CD30, GPC-3, CEA, Claudin18.2, EpCAM, GD2, MSLN, EGFR, MUC1, EGFRVIII, CD38, Trop-2, c-MET, Nectin-4, CD79b, CCK4, GPA33, HLA-A2, CLEC12A, p-cadherin, TDO2, MART-1, Pmel 17, MAGE-1, AFP, CA125, TRP-1, TRP-2, NY-ESO, PSA, CDK4, BCA225, CA 125, MG7-Ag, NY-CO-1, RCAS 1, SDCCAG16, TAAL6 and TAG72.

3. The genetically modified oHSV of claim 1, wherein the at least one chemokine is selected from a group consisting of CXCL1 to CXCL17, CCL1 to CCL 28, XCL1, XCL2, and CX3CL1.

4. The genetically modified oHSV of claim 1, wherein the truncated nonsignaling variant is an extracellular domain, an extracellular-transmembrane domain, or an equivalent thereof that has at least 90% amino acid sequence identity to the extracellular or extracellular-transmembrane domain.

5. The genetically modified oHSV of claim 1, wherein the immediate-early gene promoter of HSV is selected from a group consisting of IE 1 (ICP0 promoter), IE 2 (ICP27 promoter), IE 3 (ICP4 promoter), and IE 4/5 (ICP22 and ICP47 promoter) of HSV-1.

6. The genetically modified oHSV of claim 1, wherein the polynucleotide encodes truncated nonsignaling variants of two tumor associated/specific antigens; and at least one chemokine.

7. The genetically modified oHSV of claim 1, wherein the at least one chemokine comprises CCL5.

8. The genetically modified oHSV of claim 1, wherein the polynucleotide is inserted between UL37 and UL38.

9. The genetically modified oHSV of claim 1, wherein the polynucleotide encodes:
(a) a truncated nonsignaling variant of CD19, a truncated nonsignaling variant of BCMA, and CCL5;
(b) a truncated nonsignaling variant of CD19, a truncated nonsignaling variant of Trop-2, and CCL5;
(c) a truncated nonsignaling variant of CD19, a truncated nonsignaling variant of HER2, and CCL5;
(d) a truncated nonsignaling variant of CD19 and CCL5;
(e) a truncated nonsignaling variant of Trop-2 and CCL5;
(f) a truncated nonsignaling variant of BCMA and CCL5; or
(g) a truncated nonsignaling variant of HER2 and CCL5.

10. The genetically modified oHSV of claim 1, wherein the immediate-early gene promoter of HSV is immediate-early gene promoter IE4/5 of HSV-1.

11. The genetically modified oHSV of claim 1, wherein a PolyA tail is located downstream the polynucleotide encoding the truncated antigen and the chemokine.

12. The genetically modified oHSV of claim 1, wherein the tumor cell is a solid tumor cell.

13. The genetically modified oHSV of claim 1, wherein the tumor cell does not express the tumor associated/specific antigen encoded by the polynucleotide.

14. The genetically modified oHSV of claim 1, wherein the oHSV is further modified such that a fragment of nucleic acid of the oHSV is deleted.

15. The genetically modified oHSV of claim 14, wherein the fragment of nucleic acid of the oHSV is internal inverted repeats of the oHSV, a fragment encoding a viral gene, or both.

16. The genetically modified oHSV of claim 14, wherein the fragment of nucleic acid of the oHSV is from positions 117005 to 132096 in the P prototype genome of F strain.

17. The genetically modified oHSV of claim 1, wherein the oHSV is further modified to encode an immunostimulatory agent, an immunotherapeutic agent, or both.

18. The genetically modified oHSV of claim 17, wherein the immunostimulatory agent is selected from a group consisting of GM-CSF, IL-2, IL-12, IL-15, IL-24, and IL-27.

19. The genetically modified oHSV of claim 17, wherein the immunotherapeutic agent is an anti-PD-1 antibody, an anti-CTLA4 antibody, or an antigen binding fragment thereof.

20. The genetically modified oHSV of claim 17, wherein the immunostimulatory agent is IL-12 and the immunotherapeutic agent is an anti-PD-1 antibody or an antigen binding fragment thereof.

21. A pharmaceutical kit for treatment of a cancer, comprising, separately, an oHSV of claim 1, and a tumor-targeting therapeutic agent, wherein the tumor-targeting therapeutic agent has a targeting moiety specific to the truncated nonsignaling variant of the at least one tumor associated/specific antigen encoded by the polynucleotide, and an effector moiety for killing or inhibiting the proliferation of a cell of the cancer.

22. The pharmaceutical kit of claim 21, wherein the tumor-targeting therapeutic agent is selected from a group consisting of CAR-T cell, CAR-NK cell, BiTE and ADC.

23. The pharmaceutical kit of claim 22, wherein the tumor-targeting therapeutic agent is selected from a group consisting of a CD19-targeted CAR-T cell, a CD19-targeted CAR-NK cell, and a CD19- or EpCAM-targeted BiTE.

24. The pharmaceutical kit of claim 22, wherein the tumor-targeting therapeutic agent is a HER2-, Trop-2-, Nectin-4-, BCMA-, CD33-, CD30-, CD22-, or CD79b-targeted ADC.

25. The pharmaceutical kit of claim 22, wherein the tumor-targeting therapeutic agent is selected from a group consisting of Tecartus®, Kymriah®, Yescarta®, JWCAR-029, IM19CAR-T, CNCT19, BZ019, HD CD19 CAR-T, pCAR-19B, CD19-CART, CT032, iPD1 CD19 eCAR-T, LCAR-B38M, CT103A, CAR-BCMA T, AU-101, 4SCAR-PSMA, PSMA-CART, P-PSMA-101, C-CAR066, MB-CART20.1, PBCAR20A, LB1095, LB1901, PRGN-3006, AMG553, CT041, CD30.CAR-T, and CAR-GPC3 T.

26. The pharmaceutical kit of claim 22, wherein the tumor-targeting therapeutic agent is selected from a group consisting of Blincyto®, AMG420, PF-3135, and GBR1302.

27. The pharmaceutical kit of claim 22, wherein the tumor-targeting therapeutic agent is selected from a group consisting of Kadcyla®, Enhertu®, SHR-A1811, TAA013, RC-48, BAT8001, ARX788, A166, Trodelvy®, BAT8003, DAC-002, DS-1062, SKB264, RC-108, TR1801-ADC, Padccv®, Polivy®, Adcetris®, Mylotarg®, Blenrep®, PSMA ADC, ADCT-402, PTK7-ADC, and TRS005.

28. A genetically modified oncolytic herpes simplex virus (oHSV), wherein the genome of the oHSV is incorporated with a polynucleotide encoding a truncated nonsignaling variant of at least one tumor associated/specific antigen, wherein the expression of the truncated nonsignaling variant is under the control of an immediate-early gene promoter of HSV, and wherein the truncated nonsignaling variant is expressed and presented on a tumor cell surface as a biomarker upon replication of the oHSV in the tumor cell.

29. A pharmaceutical kit for treatment of a cancer, comprising, separately, an oHSV of claim 28, and a tumor-targeting therapeutic agent, wherein the tumor-targeting therapeutic agent has a targeting moiety specific to the truncated nonsignaling variant of the at least one tumor associated/specific antigen encoded by the polynucleotide, and an effector moiety for killing or inhibiting the proliferation of a cell of the cancer.


SEQUENCE LISTING (17 sequences)

SEQ ID NO: 1 - DNA, 63 bp, Artificial Sequence
  primer
ggaagcggagagggcagaggaagtctgctaacatgcggtgacgtcgaggagaatcctggacct

SEQ ID NO: 2 - DNA, 31 bp, Artificial Sequence
  primer
gaagatctaatatttttattgcaactccctg

SEQ ID NO: 3 - DNA, 31 bp, Artificial Sequence
  primer
ctagctagcttataaaaggcgcgtcccgtgg

SEQ ID NO: 4 - DNA, 25 bp, Artificial Sequence
  primer
gctctagattgcgacgccccggctc

SEQ ID NO: 5 - DNA, 35 bp, Artificial Sequence
  primer
ccttaattaaggttaccaccctgtagccccgatgt

SEQ ID NO: 6 - DNA, 31 bp, Artificial Sequence
  primer
tcccatggatttaacaaacgggggggtgtcg

SEQ ID NO: 7 - DNA, 28 bp, Artificial Sequence
  primer
ggcccccgaggccagcatgacgttatct

SEQ ID NO: 8 - DNA, 32 bp, Artificial Sequence
  primer
gagtaaccgccccccccccatgccaccctcac

SEQ ID NO: 9 - DNA, 31 bp, Artificial Sequence
  primer
gtgttttactgccactacacccccggggaac

SEQ ID NO: 10 - DNA, 31 bp, Artificial Sequence
  primer
tctagagatcagggtgttgatcaccacggag

SEQ ID NO: 11 - DNA, 31 bp, Artificial Sequence
  primer
gaattcgggctgcccgcacatccggacaatc

SEQ ID NO: 12 - DNA, 32 bp, Artificial Sequence
  primer
catatgggacggcgtgggttgcggactttctg

SEQ ID NO: 13 - DNA, 32 bp, Artificial Sequence
  primer
ttaattaatcacgcgcatgcccgccactcgcc

SEQ ID NO: 14 - protein, 323 aa, Artificial Sequence
  truncated nonsignaling variant of CD19
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 15 - protein, 77 aa, Artificial Sequence
  truncated nonsignaling variant of BCMA
MLQMAGQCSQNEYFDSLLHACIPCQLRCSSNTPPLTCQRYCNASVTNSVKGTNAILWTCLGLSLIISLAVFVLMFLL

SEQ ID NO: 16 - protein, 675 aa, Artificial Sequence
  truncated nonsignaling variant of HER2
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILI

SEQ ID NO: 17 - protein, 297 aa, Artificial Sequence
  truncated nonsignaling variant of Trop-2
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVI